(19) United States
(12) Patent Application Publication
(10) Pub. No.: US 2025/0035650 A1
(43) Pub. Date: Jan. 30, 2025

(54) BIOMARKER TESTING FOR CHRONIC PERSISTENT INJURY FOLLOWING BRAIN TRAUMA

(71) Applicant: BRAINBOX SOLUTIONS, INC., Richmond, VA (US)

(72) Inventors: Donna J. EDMONDS, Richmond, VA (US); Timothy E. VAN METER, Richmond, VA (US); Nazanin MIRSHAHI, Richmond, VA (US)

(21) Appl. No.: 18/710,399

(22) PCT Filed: Nov. 22, 2022
(86) PCT No.: PCT/US2022/080374

§ 371 Date: May 15, 2024

Related U.S. Application Data
(60) Provisional application No. 63/282,176, filed on Nov. 22, 2021.
(60) Provisional application No. 63/282,619, filed on Nov. 23, 2021.

(51) Int. Cl. G01N 33/68; G01N 33/86

(52) U.S. Cl. CPC G01N 33/6896; G01N 33/6848; G01N 33/86; G01N 2800/28

(57) ABSTRACT

Methods, compositions and kits useful in the assessment and monitoring post traumatic brain injury (TBI) symptoms, including neurodegenerative and/or vascular dysfunction, based on detecting changes in measured blood levels of vWF, NRGN, FABP7, GFAP, BDNF, NSE and ST2.

### CROSS REFERENCE TO RELATED APPLICATIONS

This application claims priority to U.S. Provisional Application No. 63/282,176, filed on Nov. 22, 2021; and U.S. Provisional Application No. 63/282,619, filed on Nov. 23, 2021, the disclosures of which are incorporated by reference.

### BACKGROUND OF THE INVENTION

Brain injury can occur due to an acute blunt force trauma, or from intrinsic damage due to hemorrhage, or a degenerative process in the brain. Traumatic brain injury, or TBI, has historically been studied as an acute event of force-related damage to the brain, but with the understanding that lasting symptomatic deficits can result. Differences in patient outcome are difficult to predict due to intrinsic differences in the health and genetic constitution of individuals who suffer a TBI. The critical indicators of recovery are not known and are poorly understood. Biomarker levels in biofluids could provide an objective means of identifying patient recovery profiles and could be used to monitor individuals throughout the care continuum after TBI, including the care of a subset of TBI cases that have chronic pathologies and symptoms.

Chronic TBI, which is generally recognized in the field as being present when symptoms persist more than about 3 months after a TBI event, is particularly difficult to diagnose and/or treat. For example, current methods for monitoring ongoing changes in brain pathology following a traumatic brain injury (TBI) require TBI patients to undergo multiple highly technical, time-consuming, and expensive neuroimaging procedures.

### SUMMARY OF THE INVENTION

Applicant recognized that chronic TBI may be associated with changes in the brain that indicate ongoing neurodegenerative processes or brain vascular dysfunction, including related processes that underly many common long-term symptoms, including important links to neuropsychiatric changes, seizure activity, and sleep dysfunction. Applicant discovered that certain brain biomarkers remain elevated or decreased in certain patients and correlate with symptoms and neurovascular damage associated with chronic TBI. More specifically, changes in levels of such biomarkers, including, for example, Neurogranin (NRGN), von Willebrand Factor (vWF), Brain-Derived Neurotrophic Factor (BDNF), Fatty acid binding protein 7 (FABP7), Glial Fibrillary Acidic Protein (GFAP), Neuron Specific Enolase (NSE), and Suppressor of Tumorigenesis 2 (ST2) in biological samples obtained from subjects that have experienced TBI, as compared to levels of the same biomarkers in control samples or samples obtained at earlier TBI stages, may correspond with ongoing neurodegenerative pathology and/or ongoing neurovascular dysfunction indicative of chronic TBI.

Methods, compositions, and kits made according to the principles and illustrative embodiments of the invention offer clinical and medical advantages and improvements for identifying, diagnosing, and/or assessing neurological injury and/or disease over time following a traumatic brain injury, especially for chronic TBI. For example, embodiments of the invention may obviate the need for TBI patients to undergo multiple highly technical, time-consuming, and expensive neuroimaging procedures. Accordingly, the methods, compositions, and kits made according to the principles and illustrative embodiments of the invention can provide detection and treatment benefits for patients having neurological injuries such as brain injury, including over the course of brain injury progression.

Methods, compositions, and kits made according to the principles and illustrative embodiments of the invention are capable of detecting, identifying, diagnosing, prognosing, assessing, monitoring, and/or treating a neurological injury such as a TBI and involve the use of newly-discovered correlations of changes in post-TBI pathology to one or more subsets of protein biomarkers. In particular, and as described in more detail in one or more of the embodiments disclosed herein, Applicant has determined that post-TBI changes in levels of the biomarker von Willebrand Factor (vWF) in combination with one or more of the biomarkers Neurogranin (NRGN), Brain Derived Neurotrophic Factor (BDNF) and Fatty acid binding protein 7 (FABP7) in an individual over time correspond to chronic changes in brain pathology.

According to one aspect of the invention, methods determine ongoing neurodegenerative and/or ongoing neurovascular dysfunction in a subject known to have had or suspected of having had a traumatic brain injury (TBI) event, the method comprising: (A) obtaining a biological sample from the subject; (B) measuring the level of the biomarker, von Willebrand Factor (vWF), and one or more of the following biomarkers in the biological sample: (i) Neurogranin (NRGN); (ii) Brain Derived Neurotrophic Factor (BDNF); and (iii) Fatty acid binding protein 7 (FABP7); and; (C) determining ongoing neurodegenerative dysfunction and/or ongoing neurovascular dysfunction in the subject when the measured levels of vWF and one or more of the additional biomarkers are altered relative to respective reference levels of the biomarkers.

Ongoing neurodegenerative dysfunction may be determined when the measured level of NRGN is increased and at least one of BDNF and FABP7 is decreased relative to the respective reference levels of same one or more biomarkers.

Ongoing neurovascular dysfunction may be determined to be indicative of chronic TBI based upon the time period after injury in which the sample was obtained.

The reference levels of the biomarkers for determining ongoing neurodegenerative and/or ongoing neurovascular dysfunction when respective reference levels of the biomarkers may be determined using a biological sample obtained from the same subject about three (3) months, months prior to obtaining the biological sample in step (A), thereby indicating chronic TBI.

The reference levels may be determined within 24 hours of the traumatic brain injury event that the subject known to have had or suspected of having had.

Ongoing neurodegenerative and/or ongoing neurovascular dysfunction may be determined when a ratio of the level of vWF and the level of one or more of the additional biomarkers in the biological sample to the reference level of vWF and the level of the same one or more additional biomarkers is more than about 1.

Ongoing neurodegenerative and/or ongoing neurovascular dysfunction may be determined when of the level of vWF and the level of one or more of the additional biomarkers in the biological sample to the reference level of vWF and the reference level of the same one or more additional biomarkers is more than about 2 or more than about 3.

The method may further include administering treatment for a brain injury when altered levels of vWF and one or both of NRGN and BDNF are detected.

According to another aspect of the invention, methods monitor a neurodegenerative and/or neurovascular dysfunction in a subject known to have had, or suspected of having had a traumatic brain injury (TBI), the method comprising: (A) obtaining first and second biological samples from the subject at first and second timepoints, respectively, wherein the second timepoint is after the first timepoint; (B) measuring the level of the biomarker, von Willebrand Factor (vWF), and one or more of the following biomarkers in the biological sample: (i) Neurogranin (NRGN); (ii) Brain Derived Neurotrophic Factor (BDNF); and (iii) Fatty acid binding protein 7 (FABP7); and (C) administering treatment for TBI to the subject when the measured level of vWF and one or more of the additional biomarkers are altered in the second sample relative to the first sample.

The method may administer treatment for TBI when the measured level of vWF is increased and the measured level of NRGN is increased.

The method may administer treatment for TBI when the measured level of vWF is increased and the measured level of one or more of the additional biomarkers is decreased.

An ongoing neurodegenerative dysfunction may be monitored when the measured levels of two or more of NRGN, BDNF, and FABP7 are increased in the second sample relative to the levels of the same biomarkers in the first sample.

An ongoing neurovascular dysfunction may be determined to be indicative of chronic TBI based upon the time period after injury in which the sample was obtained.

The levels of the biomarkers in the first biological sample for monitoring ongoing neurodegenerative and/or ongoing neurovascular dysfunction may be obtained about 3 months, about 4 months, about 5 months, about 6 months, about 7 months, about 8 months, about 9 months, about 10 months, about 11 months, or about 12 months prior to obtaining the second biological sample in step (A) from the same subject, thereby indicating chronic TBI.

The first timepoint may be within 24 hours of the traumatic brain injury event that the subject known to have had or suspected of having had.

Ongoing neurodegenerative and/or ongoing neurovascular dysfunction may be monitored when a ratio of the level of the level of vWF and the level of one or more of the additional biomarkers in the second sample to the levels of the same biomarkers in the first sample is more than about 1

Ongoing neurodegenerative and/or ongoing neurovascular dysfunction may be monitored when a ratio of the level of vWF and the levels of one or more of the additional biomarkers in the second sample to the levels of the same biomarkers in the first sample is more than about 2 or more than about 3.

The method may further include imaging the brain of the subject by dynamic contrast enhanced MRI (DCE-MRI) to generate normalized permeability index (NPI) values on or about the same days as the first and second timepoints, and administering treatment for TBI when the biomarker value generated on or about the second timepoint is elevated relative to the biomarker value generated on or about the first timepoint.

According to another aspect of the invention, methods monitor a secondary brain injury in a subject known to have had, or suspected of having had a traumatic brain injury (TBI), the method comprising: (A) obtaining first and second biological samples from the subject at first and second timepoints, respectively, wherein the second timepoint is after the first timepoint; (B) measuring the level of the biomarker, von Willebrand Factor (vWF), and one or more of the additional biomarkers in the biological sample: (i) Neurogranin (NRGN); (ii) Brain Derived Neurotrophic Factor (BDNF); and (iii) Fatty acid binding protein 7 (FABP7); and (C) administering treatment for TBI to the subject when the measured levels of vWF and one or more of the additional biomarkers are altered in the second sample relative to the first sample.

The method may administer treatment for TBI when the measured level of vWF is increased and the measured level of NRGN is increased.

The method may administer treatment for TBI when the measured level of vWF is increased and the measured level of one or more of the additional biomarkers is decreased. Ongoing neurodegenerative dysfunction may be determined when monitoring a secondary brain injury when measured levels of NRGN are increased, and BDNF are decreased in the second sample relative to the levels of the same biomarkers in the first sample.

An ongoing neurovascular dysfunction may be determined to be indicative of chronic TBI based upon the time period after injury in which the second sample was obtained relative to the time the first sample was obtained when monitoring a secondary brain injury.

The levels of the biomarkers in the first biological sample for monitoring a secondary brain injury may be obtained about 3 months, about 4 months, about 5 months, about 6 months, about 7 months, about 8 months, about 9 months, about 10 months, about 11 months, or about 12 months prior to obtaining the second biological sample in step (A) from the same subject, thereby indicating chronic TBI.

The first timepoint may be within 24 hours of the secondary traumatic brain injury that the subject known to have had or suspected of having had.

The method of monitoring a secondary brain injury may further include administering treatment for TBI when a ratio of the levels of vWF and one or more of the additional biomarkers in the second sample to the levels of vWF and the same one or more additional biomarkers in the first sample is more than about 1.

The method of monitoring a secondary brain injury may further include administering treatment for TBI when a ratio of the levels of vWF and one or more of the additional biomarkers in the second sample to the levels of vWF and the same one or more additional biomarkers in the first sample is more than about 2 or more than about 3.

The measuring step, (B), of the method may further include measuring the levels of one or more of the biomarkers Glial Fibrillary Acidic Protein (GFAP), Neuron Specific Enolase (NSE), and Suppressor of Tumorigenesis 2 (ST2) in the biological sample and determining differences in the levels of the one or more biomarkers relative to respective reference levels of the biomarkers, when determining ongoing neurodegenerative and/or ongoing neurovascular dysfunction.

The measuring step, (B), may further include measuring the level of one or more of the biomarkers Glial Fibrillary Acidic Protein (GFAP), Neuron Specific Enolase (NSE), and Suppressor of Tumorigenesis 2 (ST2) in the first and second biological samples and determining a difference in the level of the one or more biomarkers in the second sample relative to the level of the same one or more marker in the first sample, when monitoring a neurodegenerative and/or ongoing neurovascular dysfunction or a secondary brain injury.

The measuring step, (B), of the method may be carried out by: (i) contacting the biological sample with a plurality of antibodies or antibody fragments specific for vWF and a plurality of antibodies or antibody fragments specific for one or more of the biomarkers NRGN, BDNF, and FABP7, respectively; and (ii) detecting binding of the vWF-specific antibodies or antibody fragments specifically bound to vWF and detecting binding of the one or more additional biomarker-specific antibodies or antibody fragments specifically bound to one or more additional biomarker in the sample to measure the levels of the biomarkers.

The measuring step, (B), of the method may further include: (i) contacting the biological sample with a plurality of antibodies or antibody fragments specific for one or more of the biomarkers GFAP, NSE, and ST2, respectively; and (ii) detecting binding of antibodies or antibody fragments specifically bound to the biomarkers in the sample to measure the levels of the biomarkers.

Detecting binding of the antibodies or antibody fragments in the method may be carried out by an immunoassay, an immunoblotting method, an immunoprecipitation assay, an immunostaining method, a quantitative assay, an immunofluorescent assay, or a chemiluminescence assay.

The measuring step, (B), of the method may be carried out by using mass spectroscopy to measure levels of one or more

The biological samples used in the method may be blood, plasma, serum, cerebrospinal fluid (CSF), saliva, or tissue.

### DESCRIPTION OF THE FIGURES

FIG. 1 shows serum levels of GFAP detected in subjects with Acute TBI within 24 hours of trauma and at 6 months compared to Healthy and Trauma Controls. P-value from F-test comparing Healthy Controls, Trauma Controls, Acute TBI <0.0001. Paired t-test comparing Acute mTBI and 6-months post-TBI (p-value=0.003). T-test comparing independent subjects at Acute TBI and 6-months post-TBI (p-value <0.0001).

FIG. 2 shows serum levels of BDNF detected in subjects with Acute TBI within 24 hours of trauma and at 6 months post-TBI compared to Healthy and Trauma Controls. P-value from F-test comparing Healthy Controls, Trauma Controls, Acute TBI (p<0.0001). Paired t-test comparing Acute mTBI and 6-months post-TBI (p-value=0.0001). T-test comparing independent subjects at Acute TBI and 6-months post-TBI TBI (p-value <0.0004).

FIG. 3 shows serum levels of NSE detected in subjects with Acute TBI within 24 hours of trauma and at 6 months compared to Healthy and Trauma Controls. P-value from F-test comparing Healthy Controls, Trauma Controls, Acute TBI (p<0.0001). Paired t-test comparing Acute mTBI and 6-months post-TBI (p-value=0.0001). T-test comparing independent subjects at Acute TBI and 6-months TBI (p-value <0.13).

FIG. 4 shows serum levels of NRGN detected in subjects with Acute TBI within 24 hours of trauma and at 6 months post-TBI compared to Healthy and Trauma Controls. (P-value from F-test comparing Healthy Controls, Trauma Controls, and Acute TBI (p<0.0001). Paired t-test comparing Acute mTBI and 6-months post-TBI (p-value=0.0001). T-test comparing independent subjects at Acute TBI and 6-months after TBI (p-value <0.04).

FIG. 5 shows serum levels of vWF detected in subjects with Acute TBI within 24 hours of trauma and at 6 months compared to Healthy and Trauma Controls. P-value from F-test comparing Healthy Controls, Trauma Controls, Acute TBI <0.05. Paired t-test comparing Acute mTBI and 6-months TBI (p-value=0.0001). T-test comparing independent subjects at Acute TBI and 6-months TBI (p-value <0.0001).

FIG. 6 shows serum levels of ST2 detected in subjects with Acute TBI within 24 hours of trauma and at 6 months compared to Healthy and Trauma Controls. P-value from F-test comparing Healthy Controls, Trauma Controls, Acute TBI <0.0001. Paired t-test comparing Acute mTBI and 6-months TBI (p-value=0.0001). T-test comparing independent subjects at Acute TBI and 6-months TBI (p-value <0.0001).

FIG. 7 shows serum levels of four Severe TBI biomarkers measured during the first 24 hours after injury, compared with uninjured healthy control subjects. Elevations (GFAP, NSE) or decreases (BDNF, SNCB) shown in serum biomarker levels after severe TBI (n=67). Control subjects (n=250) represent a baseline level in the normal population. Red boxes, healthy control subjects: blue boxes, Severe TBI subjects. Black lines, median value for each distribution.

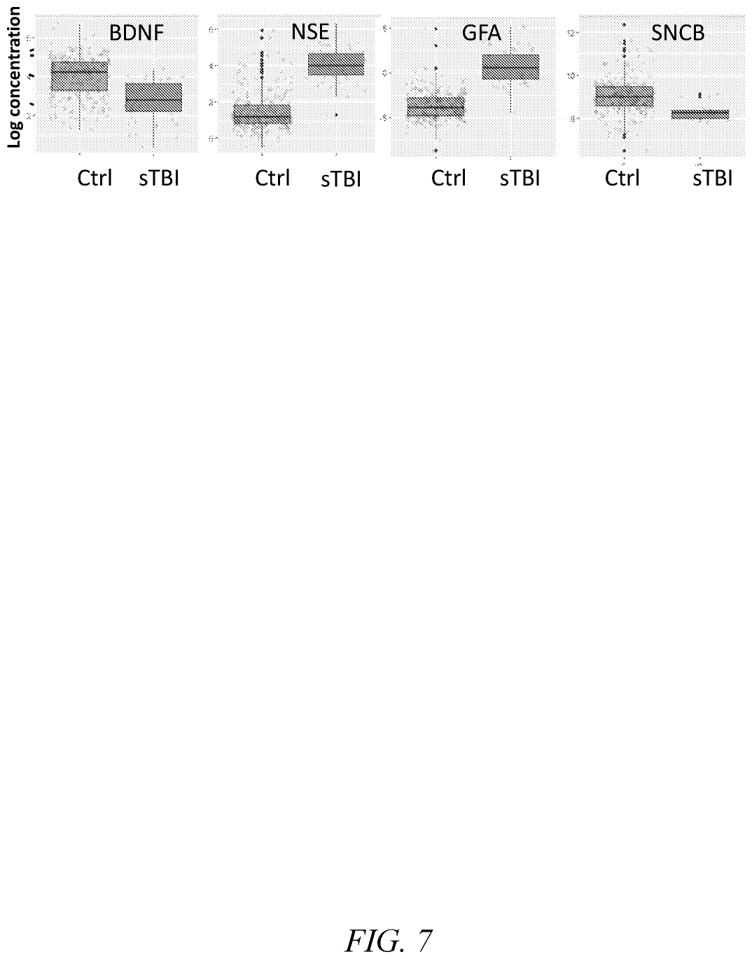

FIG. 8 shows serum levels of TBI biomarkers measured during the first 24 hours after injury, compared across 3 independent cohorts differing in severity. Elevations of biomarkers correlate with injury severity. Mild (mild TBI based on GCS 13-15); Mod (moderate TBI based on GCS 8-12); Severe (severe TBI based on GCS <8. For mild: Left box plots, CT negative, righthand box plots CT positive (abnormal). All moderate and Severe samples are by definition CT Positive.

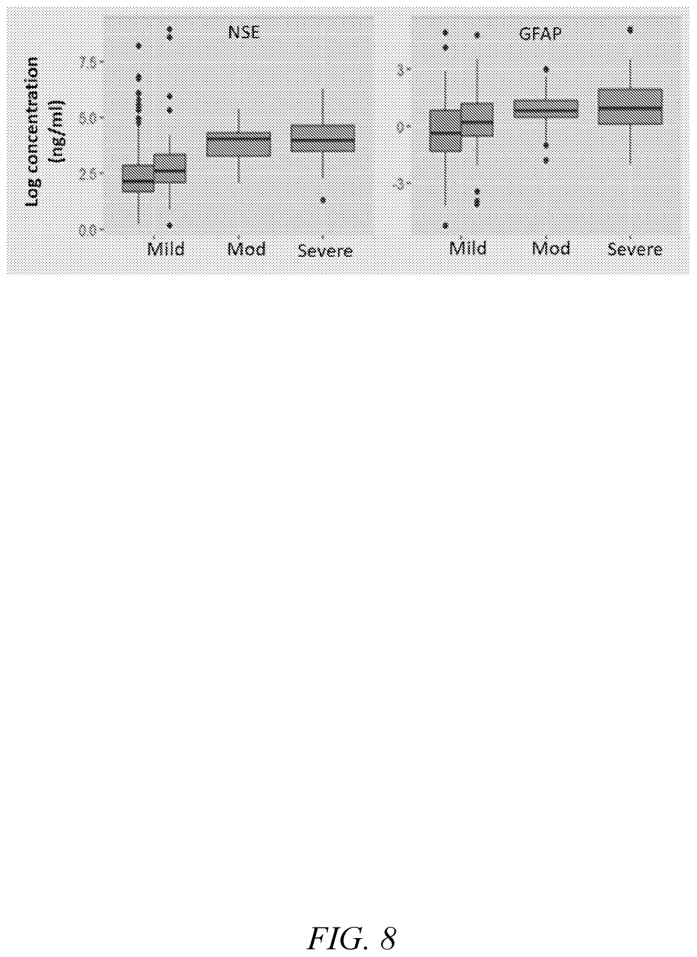

FIG. 9 shows longitudinal changes in serum biomarker levels in individual sTBI patients. Longitudinal plots of serum biomarker levels in individual Gainesville sTBI patients, measured by MSD assay across 10 days following injury. GFAP shows secondary peaks that may indicate secondary injury, whereas FABP7 decreases after injury but rises in a diurnal pattern during recovery.

FIG. 10 shows bar graphs of ratios of selected biomarker blood levels collected from chronic TBI (6 months post-TBI) cases compared to the blood levels of the same biomarkers collected from the same individuals during the acute phase of the TBI (<3 days).

### DETAILED DESCRIPTION OF THE INVENTION

The methods, compositions, and kits described herein are based on the discovery that changes in blood levels of certain protein biomarkers over time following a traumatic brain injury (TBI) correspond to ongoing TBI pathology.

TBI is an injury to the head that typically involves an acute mechanical event, in which sheer force, blunt force, or linear acceleration or deceleration damages brain tissue. Those having skill in the art appreciate that even individuals who are completely asymptomatic after a head injury can have symptoms or disabilities that develop over time, such as weeks to months after the initial injury. Late emerging deficits in patients can also result from multiple subclinical or sub-concussive head injuries.

For example, to follow the course of neural inflammation and subsequent degeneration or repair mechanisms in patients who have or are suspected of having TBI, biological samples from the patients are examined at several time points after the patient experiences or presents with TBI. Certain protein biomarkers are detected in elevated (increased), acutely elevated, or decreased amounts, levels, or concentrations in the patient's sample as well as are biomarkers that are involved with chronic degradative processes in the patient. Thus, the methods in which these protein biomarkers are detected allow for determining the evolution of post-TBI responses and for arriving at an accurate molecular and anatomical picture of TBI in a patient across a given time course.

### Definitions

The meaning of certain terms and phrases employed in the specification, examples, and appended claims are provided. The definitions are not meant to be limiting in nature and are intended to provide a clearer understanding of certain aspects and embodiments of the invention.

The term “about” as used herein means, in quantitative terms, plus or minus 5%, or in another embodiment, plus or minus 10%, or in another embodiment, plus or minus 15%, or in another embodiment, plus or minus 20%.

The term “one or more of” refers to combinations of various biomarkers. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . to N, where “N” is the total number of protein biomarkers in the particular embodiment. The term also encompasses at least 1, at least 2, at least 3, at least 4, at least 5, at least 6, at least 7, at least 8, at least 9, at least 10, at least 11, at least 12, at least 13, at least 15, 16, 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . to N. It is understood that the recitation of biomarkers herein includes the phrase “one or more of” the biomarkers and, in particular, includes the “at least 1, at least 2, at least 3” and so forth language in each recited embodiment of a biomarker panel.

“Altered” as used herein can refer to an increase or decrease. An increase is any positive change, e.g., by at least about 5%, 10%, or 20%; by at least about 25%, 50%, 75%, or even by 100%, 200%, 300% or more, including values between the stated percentages. A decrease is a negative change, e.g., a decrease by at least about 5%, 10%, or 20%; by at least about 25%, 50%, 75%; or even an increase by 100%, 200%, 300% or more, including values between the stated percentages.

As used herein, the terms “comparing”, or “comparison” refers to assessing how the proportion, level or cellular localization of one or more biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard or control sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels/ratios that correspond to, for example, a patient having a neurological injury or brain injury, not having a neurological injury or brain injury, is responding to treatment for a neurological injury or brain injury, is not responding to treatment for the neurological injury or brain injury, is/is not likely to respond to a particular treatment for the neurological injury or brain injury, or having/not having another disease or condition. In one embodiment of the invention, the term “comparing” refers to assessing whether the level of one or more biomarkers of embodiments of the invention in a sample from a patient is the same as, more or less than, different from or other otherwise corresponds (or not) to levels/ratios of the same biomarkers in a control sample (e.g., predefined levels/ratios that correlate to healthy individuals, to individuals with no neurological injury or brain injury, to individuals with a lesser degree of neurological injury or brain injury, standard brain injury levels/ratios, etc.). In another embodiment of the invention, the terms “comparing”, or “comparison” refers to assessing how the proportion, level or cellular localization of one or more biomarkers in a sample from a patient relates to the proportion, level or cellular localization of another biomarker in the same sample. For example, a ratio of one biomarker to another from the same patient sample can be compared.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient is improving, not improving, etc. In specific embodiments of the invention, the parameter may include the level of one or more biomarkers as described herein. A particular set or pattern of the amounts of one or more biomarkers may indicate that a patient has improved or worsened.

The term “brain injury” refers to a condition in which the brain (central nervous system or neurological system) is damaged by injury caused by an event. As used herein, an “injury” is an alteration in cellular or molecular integrity, activity, level, robustness, state, or other alteration that is traceable to an event. For example, an injury includes a physical, mechanical, chemical, biological, functional, infectious, or other modulator of cellular or molecular characteristics. An event can include a physical trauma such as a single or repetitive impact (percussive) or a biological abnormality such as a stroke resulting from either blockade or leakage of a blood vessel. An event is optionally an infection by an infectious agent. A person of skill in the art recognizes numerous equivalent events that are encompassed by the terms “injury” and “event”.

More specifically, the term “brain injury” refers to a condition that results in central nervous system damage, irrespective of its pathophysiological basis. Among the most frequent origins of a “brain injury” are stroke and traumatic brain injury (TBI).

The term “brain injury” also refers to subclinical brain injury, spinal cord injury, and anoxic-ischemic brain injury. The term “subclinical brain injury” (SCI) refers to brain injury without overt clinical evidence of brain injury. A lack of clinical evidence of brain injury when brain injury exists could result from degree of injury, type of injury, level of consciousness, medications particularly sedation and anesthesia.

The phrase “brain injury status” includes any distinguishable manifestation of brain injury, as the case may be, (e.g., TBI, mTBI or concussion), including not having brain injury. For example, brain injury status includes, without limitation, brain injury or non-injury in a patient, the stage or severity of brain injury, the progress of brain injury (e.g., progress of brain injury over time), or the effectiveness or response to treatment of brain injury (e.g., clinical follow up and surveillance of brain injury after treatment). Based on this status, further procedures may be indicated, including additional diagnostic tests or therapeutic procedures or regimens.

The term “traumatic brain injury” or “TBI” refers to traumatic injuries to the brain which occur when physical trauma causes brain damage. For example, TBI can result from a closed head injury or a penetrating head injury. Symptoms of TBI can be mild (even imperceptible at first) and include headache, confusion, visual disturbances, and nausea. Signs of severe TBI include loss of consciousness exceeding six hours, convulsions, dilation of the pupils, and dizziness. TBI is graded as mild (mild TBI or “mTBI”) meaning a brief change in mental status or consciousness), moderate, or severe (meaning an extended period of unconsciousness or amnesia after the injury) on the basis of the level of consciousness or Glasgow coma scale (GCS) score after resuscitation. The GCS scores eye opening (spontaneous=4, to speech=3, to pain=3, none=1), motor response (obeys=6, localizes=5, withdraws=4, abnormal flexion=3, extensor response=2, none=1), and verbal response (oriented=5, confused=4, inappropriate=3, incomprehensible=2, none=1). Mild TBI (GCS 13-15) is in most cases a concussion and there is full neurological recovery, although many of these patients have short-term memory and concentration difficulties. In moderate TBI (GCS 9-13) the patient is lethargic or stuporous, and in severe injury (GCS 3-8) the patient is comatose, unable to open his or her eyes or follow commands.

“Acute TBI” refers to the period of TBI that extends from the time of injury through about the first 3 days.

“Post-acute TBI” refers to the period of TBI that extends from the acute period days and up to about one month after injury.

“Chronic TBI” chronic period is generally understood in the TBI field to begin at least about 3 months after injury, when initial symptoms should have resolved. Persisting injury related symptoms and processes are then considered to be in the chronic period after injury when tested at 6 months, as presented in this application.

A “non-traumatic brain injury” refers to brain injuries that do not involve ischemia or external mechanical force (e.g., stroke, Alzheimer's disease, Parkinson's disease, Huntington's disease, multiple sclerosis, amyotrophic lateral sclerosis, brain hemorrhage, brain infections, brain tumor, among others).

The term “mild traumatic brain injury (mTBI)” is also commonly known as “concussion” and refers to the occurrence of injury to the head or brain arising from blunt trauma or impact, or forceful motion of the head (acceleration or deceleration forces) causing one or more of the following conditions attributable to head injury: transient confusion, disorientation, or impaired consciousness; dysfunction of memory around the time of injury; or loss of consciousness lasting less than 30 minutes. One or more of the symptoms of mTBI can last a year or more following the initial head or brain injury. While early mTBI symptoms may appear to be mild, they can lead to significant, life-long impairment in an individual's ability to function physically, cognitively and psychologically. While the term “concussion” is used interchangeably with mTBI at times, concussions cover a clinical spectrum and may occur without loss of consciousness. Mild concussion may be present even if there is no external sign of trauma to the head. The spectrum of concussions related to sports injuries are defined by The Quality Standards Subcommittee of the American Academy of Neurology as follows: Grade 1 concussion: transient confusion, no loss of consciousness and duration of mental status abnormalities on examination that resolve in less than 15 minutes; Grade 2 concussion: transient confusion, no loss of consciousness, concussion symptoms or mental status abnormalities on examination that last more than 15 minutes; and Grade 3 concussion: any loss of consciousness, either brief (seconds) or prolonged (minutes). (Centers for Disease Control and Prevention).

As used herein, “secondary brain trauma” refers to damage to the brain of a patient post-acute brain injury, i.e., during the secondary injury phase of a TBI.

As used herein, “acute brain injury” refers to the condition of a patient who has suffered a neurological or brain injury and at a relatively short number of hours, such as 1-10 hours, 1-8 hours, 1-5 hours, 2-5 hours, 3-5 hours, 4-5 hours, and the like from the actual time of the injury.

As used herein, “sub-acute brain injury” refers to the condition of a patient who has suffered a neurological or brain injury from about 2-5 days post injury.

As used herein, “chronic brain injury” refers to the condition of a patient who has suffered a neurological or brain injury from about three days post injury until at least 12 months previously, or from about 1-5 months, or about 1-3 months from the actual time of injury yet continues to present symptoms of brain injury.

As used herein, the term “biomarker” refers to a molecule that is associated either quantitatively or qualitatively with a biological change. Examples of biomarkers include polypeptides, proteins or fragments of a polypeptide or protein; and polynucleotides, such as a gene product, RNA or RNA fragment, or encoding polynucleotides; and other body metabolites. In certain embodiments of the invention, a “biomarker” means a compound (e.g., a protein) that is differentially present (i.e., increased or decreased) in a biological sample from a subject or a group consisting of subjects having a first phenotype (e.g., having a disease or condition) as compared to a biological sample from a subject or group consisting of subjects having a second phenotype (e.g., not having the disease or condition or having a less severe version of the disease or condition). A biomarker may be differentially present at any level, but is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent); or that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more. Alternatively, the differential presence of a biomarker can be characterized by a -fold change in level including, for example, a level that is decreased by 1.1-fold, at least 1.2-fold, at least 1.3-fold, at least 1.4-fold, at least 1.5-fold, at least 2.0-fold, at least 2.5-fold, at least 3.0-fold, at least 3.5-fold, at least 4.0-fold, at least 5-fold, at least 5.5-fold, at least 6-fold, at least 6.5-fold, at least 7.0-fold, at least 7.5-fold, at least 8.0-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 25-fold, at least 30-fold, at least 40-fold, or at least 50-fold; or that is increased by 1.1-fold, at least 1.2-fold, at least 1.3-fold, at least 1.4-fold, at least 1.5-fold, at least 2.0-fold, at least 2.5-fold, at least 3.0-fold, at least 3.5-fold, at least 4.0-fold, at least 5-fold, at least 5.5-fold, at least 6-fold, at least 6.5-fold, at least 7.0-fold, at least 7.5-fold, at least 8.0-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 25-fold, at least 30-fold, at least 40-fold, or at least 50-fold. A biomarker is preferably differentially present at a level that is statistically significant (e.g., a p-value less than 0.05 and/or a q-value of less than 0.10 as determined using, for example, either Welch's T-test or Wilcoxon's rank-sum Test).

The term “peptide biomarkers derived therefrom” includes the isoforms and/or post-translationally modified forms of any of the foregoing. Embodiments of the invention contemplate the detection, measurement, quantification and/or determination or other analysis of both unmodified and modified (e.g., citrullination or other post-translational modification) proteins/polypeptides/peptides, as well as autoantibodies to any of the foregoing. For example, in some embodiments of the invention, the method includes the detection, measurement, quantification and/or determination or other analysis of both unmodified and modified forms of vWF, NRGN, BDNF, FABP7, GFAP, NSE, and/or ST2.

The term “biomarker panel” refers to a collection of a plurality of biomarkers grouped together for use in the embodiments of the methods, compositions and kits of the invention. The biomarkers in the panel may be protein biomarkers, or peptide biomarkers derived therefrom. In some embodiments of the methods, compositions or kits of the invention, the protein biomarker panel includes, but is not limited to any combination of von Willebrand Factor (vWF), Neurogranin (NRGN), and Brain-Derived Neurotrophic Factor (BDNF), Fatty acid binding protein 7 (FABP7), Glial Fibrillary Acidic Protein (GFAP), Neuron Specific Enolase (NSE, also called Enolase 2), and/or soluble Suppressor of Tumorigenesis 2 (sST2 or ST2). Thus, a biomarker panel of the invention may include a plurality of biomarkers. For example, a biomarker panel may include vWF and one or more of NRGN, BDNF, and FABP7.

In other embodiments, a particular set or pattern of the amounts of one or more biomarkers may be correlated to a patient being unaffected (i.e., indicates a patient does not have brain injury). In certain embodiments, “indicating,” or “correlating,” as used according to embodiments of the invention, may be by any linear or non-linear method of quantifying the relationship between levels/ratios of biomarkers to a standard, control or comparative value for the assessment of the diagnosis, prediction of a neurological injury, brain injury or progression thereof, assessment of efficacy of clinical treatment, identification of a patient who may respond to a particular treatment regime or pharmaceutical agent, monitoring of the progress of treatment, and in the context of a screening assay, for the identification of a therapeutic for the neurological injury or brain injury.

“Magnetic resonance imaging (MRI)” of the brain is a noninvasive and painless neuroimaging test for detailed visualization and analysis that uses a magnetic field and radio waves to produce detailed images of the brain and the brain stem. Unlike a CAT scan (also called a CT scan; computed axial tomography scan), an MRI scan does not use radiation. In some cases, a dye (contrast dye) or contrast material (e.g., iodine, barium, or gadolinium) is used during the MRI to allow visualization of the brain structures (e.g., blood vessels and tissue) more clearly. For example, the dye may show blood flow and areas of inflammation or edema. In some embodiments of the invention, the method detects changed or altered blood-brain barrier permeability signals in the brain by using Dynamic Contrast Enhanced MRI (DCE-MRI). In other embodiments of the invention, 3T MRI is used to changed or altered blood-brain barrier permeability signals in the brain. In yet other embodiments of the invention, diffusion weighted tensor imaging (DTI-MRI) is used to changed or altered blood-brain barrier permeability signals in the brain.

The terms “patient,” “individual,” or “subject” are used interchangeably herein, and refer to a mammal, particularly, a human. The patient may have a mild, intermediate or severe disease or condition. The patient may be an individual in need of treatment or in need of diagnosis based on particular symptoms or personal or family history. In some cases, the terms may refer to treatment in experimental animals, in veterinary application, and in the development of animal models for disease, including, but not limited to, rodents including mice, rats, and hamsters; and primates.

The terms “measuring” and “determining” are used interchangeably throughout and refer to methods which include obtaining or providing a patient sample and/or detecting the level (or amount) of a biomarker(s) in a sample. In one embodiment, the terms refer to obtaining or providing a patient sample and detecting the level of one or more biomarkers in the sample. In another embodiment, the terms “measuring” and “determining” mean detecting the level of one or more biomarkers in a patient sample. The term “measuring” is also used interchangeably throughout with the term “detecting.” In certain embodiments, the term is also used interchangeably with the term “quantifying.”

The terms “sample,” “patient sample,” “biological sample,” “biologic sample,” “biofluid sample,” and the like, encompass a variety of sample types obtained from a patient, individual, or subject and can be used in a diagnostic, screening, or monitoring assay. The patient sample may be obtained from a healthy subject or a patient suspected of having or having associated symptoms of neurological injury or brain injury. Moreover, a sample obtained from a patient can be divided, and only a portion may be used for diagnosis. Further, the sample, or a portion thereof, can be stored under conditions to maintain sample for later analysis. The definition of “sample” specifically encompasses blood, serum, plasma, cerebrospinal fluid (CSF) and other liquid samples of biological origin, including, but not limited to, peripheral blood, cord blood, blood plasma, serum, amniotic fluid, tears, urine, saliva, stool, semen, sweat, secretions and synovial fluid. A sample also encompasses solid tissue samples, such as a biopsy specimen or cells derived therefrom, or tissue culture cells and the progeny thereof. A tissue or cell sample may be processed (e.g., homogenized, etc.) to produce a suspension or dispersion in liquid form, as discussed below. In a specific embodiment, a sample includes a blood sample. In another embodiment, a sample includes a plasma sample. In yet another embodiment, a serum sample is used. In certain embodiments, a sample includes cerebrospinal fluid.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and includes cells in culture, cell supernatants, tissue samples, organs, and the like. Samples may also include fresh-frozen and/or formalin-fixed, paraffin-embedded tissue blocks, such as blocks prepared from clinical or pathological biopsies, prepared for pathological analysis or study by immunohistochemistry. A sample may be tested immediately after collection, or it may be tested after storage at 4° C., −20° C., or −80° C. Storage times may be 24 hours, 1 week, 1 month, 1 year, 10 years or up to 30 years, depending on stability of the sample and storage conditions.

Various methodologies of the embodiments of the invention include a step that involves comparing a value, level, feature, characteristic, property, etc. to a “suitable control,” referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, a “brain injury-positive reference level” of a biomarker means a level of a biomarker that is indicative of brain injury in a subject, and a “brain injury-negative reference level” of a biomarker means a level of a biomarker that is indicative of no brain injury of in a subject.

A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., ELISA, PCR, LC-MS, GC-MS, etc.), where the levels of biomarkers may differ based on the specific technique that is used.

In one embodiment, a “suitable control” or “appropriate control” is a value, level, feature, characteristic, property, etc., determined in an individual or biological sample obtained from an individual or group of individuals, (e.g., a control or normal cell, organ, or patient, exhibiting, for example, normal traits. For example, the biomarkers of the embodiments of the invention may be assayed for levels/ratios in a sample from an unaffected individual (UI) (e.g., no brain injury) or a normal control individual (NC) (both terms are used interchangeably herein). For example, a “suitable control” or “appropriate control” can be a value, level, feature, characteristic, property, ratio, etc. determined prior to performing a therapy (e.g., brain injury treatment) on a patient or a value, level, feature, characteristic, property, ratio, etc. determined prior to disease development (e.g., a baseline test). In a further embodiment, a “suitable control” or “appropriate control” is a predefined value, level, feature, characteristic, property, ratio, etc. A “suitable control” can be a profile or pattern of levels/ratios of one or more biomarkers of embodiments of the invention that correlates to brain injury, to which a patient sample can be compared. The patient sample can also be compared to a negative control, i.e., a profile that correlates to not having brain injury.

As used herein, the term “predetermined threshold value of expression” of a biomarker refers to the level of expression of the same biomarker (expressed, for example, in ng/ml) in a corresponding control/normal sample or group consisting of control/normal samples obtained from normal, or healthy, subjects, i.e., subject who do not have brain injury. Further, the term “altered level of expression” of a biomarker in a sample refers to a level that is either below or above the predetermined threshold value of expression for the same biomarker and thus encompasses either high (increased) or low (decreased) expression levels. In particular embodiments, the biomarkers described herein are increased or decreased relative to age-matched (and/or sex-matched) controls.

The terms “specifically binds to,” “specific for,” and related grammatical variants refer to that binding which occurs between such paired species as enzyme/substrate, receptor/agonist, antibody/antigen, aptamer/target, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M. As used herein, the terms “specific binding” or “specifically binding” when used in reference to the interaction of an antibody and a protein or peptide means that the interaction is dependent upon the presence of a particular structure (i.e., the epitope) on the protein.

By “antibody” is meant any immunoglobulin polypeptide, or fragment thereof, having immunogen or antigen binding ability. As used herein, the terms “antibody fragments”, “fragment”, or “fragment thereof” refer to a portion of an intact antibody, in particular, an immunogen- or antigen-binding portion of the antibody. Examples of antibody fragments include, but are not limited to, linear antibodies; single-chain antibody molecules; Fc or Fc′ peptides, Fab and Fab fragments, and multi-specific antibodies formed from antibody fragments. In most embodiments, the terms also refer to fragments that bind an antigen of a target molecule (e.g., a protein biomarker described herein) and can be referred to as “antigen-binding fragments.” As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies that specifically binds the target antigen.

By “an effective amount” is meant the amount of a required to ameliorate the symptoms of a disease relative to an untreated patient. The effective amount of active compound(s) used to practice embodiments of the invention for therapeutic treatment of brain injury varies depending upon the manner of administration, the age, body weight, and general health of the subject. Ultimately, the attending physician or veterinarian will decide the appropriate amount and dosage regimen. Such amount is referred to as an “effective” amount.

As used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of embodiments of the invention. It is understood that embodiments of the invention are not limited to the particular methods and components, etc., described herein, as these may vary. It is also to be understood that the terminology used herein is used for the purpose of describing particular embodiments only and is not intended to limit the scope of the invention.

All publications cited herein are hereby incorporated by reference including all journal articles, books, manuals, published patent applications, and issued patents.

In some embodiments of the invention, biomarkers may be detected and/or measured by immunoassay. An immunoassay requires biospecific capture reagents/binding agents, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art.

Some embodiments of the invention provide methods for measuring one or more biomarkers and determining ongoing neurovascular dysfunction in a subject after a traumatic brain injury (TBI) event, wherein levels of one or more protein biomarkers are measured in a sample obtained from a patient or subject and determining ongoing neurodegenerative dysfunction and/or ongoing neurovascular dysfunction in the subject when the measured levels of the one or more biomarkers are increased or decreased relative to respective reference levels of the biomarkers. In some such embodiments of the invention, ongoing neurodegenerative dysfunction is determined when the measured levels of NRGN and/or BDNF are increased relative to the respective reference levels of the biomarkers. In the same or other such embodiments of the invention, ongoing neurovascular dysfunction is determined when the measured level of vWF is increased relative to the respective reference levels of vWF. For example, in one embodiment of the invention, methods are provided that include: (A) obtaining a biological sample from the subject; (B) measuring the levels of one or more of the biomarkers Neurogranin (NRGN), von Willebrand Factor (vWF), and Brain-Derived Neurotrophic Factor (BDNF) in the biological samples; and (C) determining ongoing neurodegenerative dysfunction and/or ongoing neurovascular dysfunction in the subject when the measured levels of the one or more of NRGN, vWF, and/or BDNF are increased relative to respective reference levels of the biomarkers. In the same or different embodiment of the invention, levels of one or more biomarkers GFAP, NSE, and ST2 are also measured, and ongoing neurodegenerative dysfunction and/or ongoing neurovascular dysfunction in the subject is determined when the measured levels of the one or more of GFAP, NSE, and ST2 are increased relative to respective reference levels of the biomarkers. In some embodiments of the invention, methods of measuring biomarkers include (i) contacting a biological sample with a plurality of antibodies or antibody fragments specific for the one or more biomarkers; and (ii) detecting binding of antibodies or antibody fragments specifically bound to the biomarkers in the sample to measure the levels of the biomarkers. Accordingly, in an embodiment of the invention, a method of measuring biomarkers includes (i) contacting a biological sample with a plurality of antibodies or antibody fragments specific for one or more of the biomarkers, NRGN, vWF, BDNF, GFAP, NSE, and ST2, optionally (NRGN, vWF, and BDNF) or (NRGN, vWF, BDNF, GFAP, NSE, and ST2); and (ii) detecting binding of antibodies or antibody fragments specifically bound to the biomarkers in the sample to measure the levels of the biomarkers. It is to be understood herein that biological samples are contacted with antibodies or antibody fragments for a period of time sufficient to form antibody-biomarker and/or antibody fragment-biomarker complexes.

Some embodiments of the invention provide methods for monitoring a neurodegenerative and/or neurovascular dysfunction in a subject known to have, or suspected of having an acute traumatic brain injury (TBI) levels, wherein one or more protein biomarkers are measured in first and second samples obtained from a subject at first and second timepoints, respectively, wherein the second timepoint is after the first timepoint, and administering treatment for TBI to the subject when the measured levels of one or more of NRGN, vWF, and BDNF are increased in the second sample relative to the first sample or determining the subject can return to work or play when the measured levels of one or more of NRGN, vWF, and BDNF are decreased in the second sample relative to the first sample. In an embodiment of the invention in which treatment for TBI is administered to a subject, as described above, the subject is treated when the ratio of the level of one or more of the biomarkers in the second sample to the level of the same one or more biomarkers in the first sample more than about 1, more than about 2, or more than about 3. Thus, in some embodiments of the invention, the subject is treated in a method for monitoring a neurodegenerative and/or neurovascular dysfunction in a subject, when the ratio of the level of one or more of NRGN, vWF, and BDNF in the second sample to the level of one or more of NRGN, vWF, and BDNF in the first sample is more than about 1, more than about 2, or more than about 3.

In some embodiments of the invention, ongoing neurodegenerative dysfunction is determined when the measured levels of NRGN and/or BDNF are increased in the second sample relative to the levels of the NRGN, vWF, and BDNF in the first sample. In the same or other such embodiments of the invention, ongoing neurovascular dysfunction is determined when the measured level of vWF is increased in the second sample relative to the levels of vWF in the first sample. For example, in one embodiment of the invention, methods are provided that include: (A) obtaining a biological sample from the subject; (B) measuring the levels of one or more of the biomarkers Neurogranin (NRGN), von Willebrand Factor (vWF), and Brain-Derived Neurotrophic Factor (BDNF) in the biological samples; and (C) determining ongoing neurodegenerative dysfunction and/or ongoing neurovascular dysfunction in the subject when the measured levels of one or more of NRGN, vWF, and/or BDNF are increased relative to respective reference levels of the biomarkers. In the same or different embodiment of the invention, levels of one or more biomarkers GFAP, NSE, and ST2 are also measured in the first and second samples, and ongoing neurodegenerative dysfunction and/or ongoing neurovascular dysfunction in the subject is determined when the measured levels of one or more of GFAP, NSE, and ST2 are increased in the second sample relative to respective of levels of GFAP, NSE, and ST2 in the first sample.

Some embodiments of the invention provide methods for monitoring a secondary brain injury in a subject known to have, or suspected of having a secondary traumatic brain injury (TBI), wherein one or more protein biomarkers are measured in first and second samples obtained from a subject at first and second timepoints, respectively, wherein the second timepoint is after the first timepoint, and administering treatment for the secondary TBI to the subject when the measured levels of one or more of NRGN, vWF, and BDNF are increased in the second sample relative to the first sample or determining the subject can return to work or play when the measured levels of one or more of NRGN, vWF, and BDNF are decreased in the second sample relative to the first sample. Thus, in some embodiments of the invention, the subject is treated in a method for monitoring a secondary TBI in a subject, when the ratio of the level of one or more of NRGN, vWF, and BDNF in the second sample to the level of one or more of NRGN, vWF, and BDNF in the first sample is more than about 1, more than about 2, or more than about 3.

In some embodiments of the invention, ongoing neurodegenerative dysfunction is determined when the measured levels of NRGN and/or BDNF are increased in the second sample relative to the levels of the NRGN, vWF, and BDNF in the first sample. In the same or other such embodiments of the invention, ongoing neurovascular dysfunction is determined when the measured level of vWF is increased in the second sample relative to the levels of vWF in the first sample. For example, in one embodiment of the invention, methods are provided that include: (A) obtaining a biological sample from the subject; (B) measuring the levels of one or more of the biomarkers Neurogranin (NRGN), von Willebrand Factor (vWF), and Brain-Derived Neurotrophic Factor (BDNF) in the biological samples; and (C) determining ongoing neurodegenerative dysfunction and/or ongoing neurovascular dysfunction in the subject when the measured levels of one or more of NRGN, vWF, and/or BDNF are increased relative to respective reference levels of the biomarkers. In the same or different embodiment of the invention, levels of one or more biomarkers GFAP, NSE, and ST2 are also measured in the first and second samples, and ongoing neurodegenerative dysfunction and/or ongoing neurovascular dysfunction in the subject is determined when the measured levels of one or more of GFAP, NSE, and ST2 are increased in the second sample relative to respective of levels of GFAP, NSE, and ST2 in the first sample.

Detection methods suitable for use methods of the invention, include, without limitation, traditional immunoassays including, for example, sandwich immunoassays including enzyme-linked immunosorbent assays (ELISA) or fluorescence-based immunoassays, immunoblots, Western Blots (WB), as well as other enzyme immunoassays. Multiplex ELISA assays are also suitable for use. Nephelometry is an assay performed in liquid phase, in which antibodies are in solution. The binding of a protein antigen to a specific antibody results in changes in absorbance, a parameter that is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments of the invention, the expression levels of the biomarkers employed herein are quantified by immunoassay, such as ELISA technology. In specific embodiments of the invention, the levels of expression of the biomarkers are determined by contacting the biological sample with a plurality of antibodies, or antigen binding fragments thereof, that selectively bind to the biomarkers; and detecting binding of the antibodies, or antigen binding fragments thereof, to the biomarkers. In certain embodiments, the binding agents employed in the disclosed methods and compositions are labeled with a detectable moiety.

For example, the level of a biomarker in a sample can be assayed by contacting the biological sample with an antibody, or antigen binding fragment thereof, that selectively binds to the target biomarker (referred to as a capture molecule or antibody or a binding agent), and detecting the binding of the antibody, or antigen-binding fragment thereof, to the biomarker. The detection can be performed using a second antibody to bind to the capture antibody complexed with its target biomarker. A target biomarker can be an entire protein, or a variant or modified form thereof. Kits for the detection of biomarkers as described herein can include pre-coated strip plates, biotinylated secondary antibody, standards, controls, buffers, streptavidin-horse radish peroxidase (HRP), tetramethyl benzidine (TMB), stop reagents, and detailed instructions for carrying out the tests including performing standards.

Further embodiments of the invention provide compositions that can be employed in the disclosed methods. In certain embodiments of the invention, such compositions include a solid substrate and a plurality of antibodies immobilized on the substrate, wherein each of the antibodies is immobilized at a different, indexable, location on the substrate and the antibodies selectively bind to a plurality of biomarkers in a protein biomarker panel, wherein the panel includes antibodies or antigen-binding fragments to specifically detect one or more, two or more, three or more, four or more, five or more, six or more biomarkers. For example, one or more protein biomarkers selected from GFAP, BDNF, NSE, NRGN, VWF, and ST2. Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in U.S. Patent Application Publication No. U.S. 2010/0093557 A1. Methods for performing assays employing such panels include those described, for example, in U.S. Patent Application Publication Nos. US2010/0093557A1 and US2010/0190656A1, the disclosures of which are incorporated by reference herein.

In a related aspect, methods for assessing brain injury, e.g., mTBI or concussion, in a subject are provided, such methods including: (a) contacting a biological sample obtained from the subject with a composition disclosed herein for a period of time sufficient to form binding agent-polypeptide biomarker complexes; (b) detecting binding of the plurality of binding agents to the plurality of polypeptide biomarkers in the protein biomarker panel, thereby determining the levels of expression of the plurality of polypeptide biomarkers in the biological sample; and (c) comparing the levels of expression of the plurality of polypeptide biomarkers in the biological sample with predetermined threshold values, wherein levels of expression of at least one of the plurality of polypeptide biomarkers above or below the predetermined threshold values indicates brain injury status in the subject.

Multiplex arrays in several different formats based on the utilization of, for example, flow cytometry, chemiluminescence or electron-chemiluminescence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

### Detection by Mass Spectrometry

In one embodiment of the invention, biomarkers may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap™, hybrids or combinations of the foregoing, and the like.

In some embodiments of the invention, biomarkers are detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g., 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic co-elution of multiple transitions for a given analyte. The terms SRM/MRM are occasionally used also to describe experiments conducted in mass spectrometers other than triple quadrupoles (e.g., in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application the terms SRM and MRM or also SRM/MRM can be used interchangeably because they both refer to the same mass spectrometer operating principle. As a matter of clarity, the term MRM is used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissociation (also known as CID or collision-induced dissociation), HCD (higher energy CID), ECD (electron capture dissociation), PD (photodissociation) or ETD (electron transfer dissociation).

In another embodiment of the invention, the mass spectrometric method includes matrix assisted laser desorption/ionization time-of-flight (MALDI-TOF MS or MALDI-TOF). In another embodiment, method includes MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein.

In yet another embodiment of the invention, a mass spectrometric technique includes surface enhanced laser desorption and ionization or “SELDI,” as described, for example, in U.S. Pat. Nos. 6,225,047 and 5,719,060, which are included herein in their entireties. Briefly, SELDI refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes including energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060, which is included herein in its entirety). SEPAR and other forms of SELDI are readily adapted to detecting a biomarker or biomarker panel, pursuant to the invention.

In another mass spectrometry method, the biomarkers can be first captured on a chromatographic resin having chromatographic properties that bind the biomarkers. For example, one could capture the biomarkers on a cation exchange resin, such as CM Ceramic HyperD® F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that includes antibodies that bind the biomarkers, wash the resin to remove unbound material, elute the biomarkers from the resin and detect the eluted biomarkers by MALDI or by SELDI.

### Detection by Electrochemiluminescence Assay

In some embodiments of the invention, biomarkers may be detected by means of an electrochemiluminescence assay developed by Meso Scale Discovery. Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434, which are included herein in their entireties. See also U.S. Patent Appl. Pub. No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534, which are included herein in their entireties.

### Other Methods for Detecting Biomarkers

TBI biomarkers may be detected by other suitable methods known in the art. Detection paradigms, which may be employed to this end, include optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In another embodiment of the invention, a sample, such as a sample containing the protein biomarkers described herein, may also be analyzed by means of a biochip. Biochips generally include solid substrates and have a generally planar surface, to which a capture reagent (also called an adsorbent or affinity reagent) is attached. Frequently, the surface of a biochip includes a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, CA.), Invitrogen Corp. (Carlsbad, CA), Affymetrix, Inc. (Fremont, CA), Zyomyx (Hayward, CA), R&D Systems, Inc. (Minneapolis, MN), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; PCT International Publication No. WO 2000/56934; and PCT International Publication No. WO 03/048768.

Other assays useful for detecting biomarkers include single-molecule arrays (SIMOA™), (e.g., as provided by Quanterix, Lexington, MA), which are bead-based detection assays, in which antibody capture molecules are attached to the surface of paramagnetic beads that are capable of detecting thousands of single protein molecules simultaneously and use the same reagents as are used in conventional ELISA assays described herein. Femtomolar (fg/mL) concentrations of proteins can be measured in a SIMOA bead-based immunoassay, which involves arrays of femtoliter-sized reaction chambers that can isolate and detect single protein molecules. Because the array volumes are significantly smaller than those of a conventional ELISA, a rapid increase of fluorescent product is generated if a labeled protein is present.

The power of a diagnostic test to correctly predict TBI status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments of the invention, biomarker panels may show a statistical difference in different brain injury statuses of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

The biomarkers may be differentially present in biological samples from uninjured (UI) control subjects (normal controls (NC) or non-brain injury) and biological samples from subjects with a brain injury, and, therefore, are useful in aiding in the determination of brain injury status. In some embodiments of the invention, biomarkers are measured in a patient sample using the methods described herein and compared, for example, to predefined biomarker levels/ratios and correlated to brain injury status. In particular embodiments, the measurement(s) may then be compared with a relevant diagnostic amount(s), cut-off(s), or multivariate model scores that distinguish a positive brain injury status from a negative brain injury status. The diagnostic amount(s) represents a measured amount of a biomarker(s) above which or below which a patient is classified as having a particular brain injury status. For example, if the biomarker(s) is/are up-regulated compared to normal, then a measured amount(s) above (or greater than) the diagnostic cutoff(s) provides an assessment of brain injury status. Alternatively, if the biomarker(s) is/are down-regulated, then a measured amount(s) at or below the diagnostic cutoff(s) provides an assessment of brain injury status. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the levels of biomarkers in a statistically significant number of samples from patients with the different brain injury statuses, and drawing the cut-off to suit the desired levels of specificity and sensitivity.

In other embodiments of the invention, the relative or normalized amounts of biomarkers to each other are useful in aiding in the determination of brain injury status. In certain embodiments, the biomarker ratios are indicative of diagnosis. In other embodiments, a biomarker ratio can be compared to another biomarker ratio in the same sample or to a set of biomarker ratios from a control or reference sample.

Furthermore, in some embodiments, the measured values (i.e., levels) of the biomarkers detected by a biomarker panel are mathematically combined and the combined value is correlated to the underlying diagnostic question. Biomarker values may be combined by any appropriate state of the art mathematical method. Mathematical methods useful for correlating a marker combination to a brain injury status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. In one embodiment, the method used in correlating a biomarker combination of the invention, e.g. to assess brain injury, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

### Determining Risk of Brain Injury

In some embodiments of the invention, methods are provided for determining the risk of brain injury, such as TBI, in a patient. Biomarker percentages, ratios, amounts, or patterns are characteristic of various risk states, e.g., high, medium or low. The risk of brain injury is determined by measuring the relevant biomarkers in a protein biomarker panel, and then either submitting them to a classification algorithm or comparing them with a reference amount, i.e., a predefined level or pattern of biomarkers that is associated with the particular risk level.

### Determining Severity of Brain Injury

In some embodiments of the invention, methods are provided for determining the severity of brain injury, e.g., TBI, mTBI, in a patient. Each grade or stage of brain injury likely has a characteristic level of a biomarker or relative levels/ratios of a set of biomarkers (a pattern or ratio). The severity of brain injury is determined by measuring the relevant biomarkers and then either submitting them to a classification algorithm or comparing them with a reference amount, i.e., a predefined level or pattern of biomarkers that is associated with the particular stage. In embodiments, severity of brain injury, e.g., TBI, is further determined by performing neuroimaging analysis to detect more serious or severe damage or insult, such as a change in vascular permeability, such as, for example, blood vessel leakage or intracranial hemorrhage (ICH). Neuroimaging analysis, e.g., using contrast MRI, allows for the detection and visualization of injury such as bleeding, hemorrhage, or other insult or damage to the integrity to the brain or its blood-brain barrier.

### Determining Brain Injury Prognosis

In one embodiment of the invention, methods are provided for determining the course of brain injury, e.g., TBI, mTBI or concussion, in a patient, e.g., a patient who has experienced repetitive injury. Brain injury course refers to changes in brain injury status over time, including brain injury progression (worsening) and brain injury regression (improvement). Over time, the levels, amounts, or relative levels or amounts (e.g., the pattern or ratio) of the biomarkers change. For example, biomarker “X” may be increased with brain injury, while biomarker “Y” may be decreased with brain injury. Therefore, the trend of these biomarkers, either increased or decreased over time toward neurological injury or brain injury, or recovery, indicates the course of the condition. Accordingly, in an embodiment of the invention, a method involves measuring the level of one or more biomarkers in a patient at least two different time points, e.g., at a first time point and at a second time point, and comparing the change, if any. The course of brain injury, as well as a determination of injury status, are determined based on these comparisons.

### Patient Management

In some embodiments of the invention, methods of identifying or qualifying the status of a neurological injury or a brain injury, e.g., TBI, mTBI or concussion include determining and/or managing patient treatment based on injury status and/or risk. Such management includes the decisions and actions of the medical practitioner, physician, or clinician subsequent to determining brain injury status, e.g., as to TBI, mTBI, or concussion. For example, if a physician makes a diagnosis of TBI, mTBI or concussion, then a certain monitoring regimen would follow. An assessment of the course of brain injury using the described methods may then require a certain treatment or therapy regimen. Profiles of the levels of a set of biomarkers in the biological sample, combined with the age, sex, and acute symptoms of a patient, can provide a risk stratification (high risk, lower risk, or little to no risk likelihood of developing a certain post-TBI outcome, such as seizures, chronic pain, chronic headache, post-concussive symptoms, incomplete recovery assessed by GOS-E<8, sleep disturbances, mild to severe depressive symptoms, mild to severe anxiety, PTSD, chronic headache or migraine, poor attention or cognitive performance, or motor deficits). Each model profile with these biomarkers allows the physician to better make an informed decision to direct the TBI, mild TBI, or concussion patient down a treatment pathway tailored for each of the outcomes, having determined the symptoms for which he or she is at high risk. An assessment of the course of brain injury using the described methods may then require a certain treatment or therapy regimen, including identifying an individual's eligibility for clinical trials that investigate therapeutics for a symptom or set of symptoms that results from TBI. Alternatively, a diagnosis of no brain injury might be followed with further testing or monitoring. Also, further tests may be called for if the diagnostic test gives an inconclusive result for neurological or brain injury status. In some embodiments of the invention, any of the biomarkers combinations disclosed herein may be used with any of the embodiments relating to patient management described herein

In other embodiments of the invention, a method of treating post-TBI depression includes: detecting whether a plurality of protein biomarkers in a protein biomarker panel, or peptide biomarkers derived therefrom, are present in a biological sample obtained from a patient; measuring the levels of the one or more protein biomarkers, or peptide biomarkers derived therefrom, present in the biological sample relative to the levels of the same proteins, or peptides derived therefrom, in a control sample; stratifying the risk of the patient for at one or more time points post-TBI depression when the measured levels of the one or more protein biomarkers, or peptide biomarkers derived therefrom, are increased or decreased in the subject's sample relative to the control levels; and if the risk stratification of the patient is high, administering psychotherapy or psychiatry or an effective amount of an antidepressant, such as Prozac or Elavil to the patient.

In another embodiment of the invention, a method of treating post-TBI anxiety includes: detecting whether a plurality of protein biomarkers in a protein biomarker panel, or peptide biomarkers derived therefrom, are present in a biological sample obtained from a patient; measuring the levels of the one or more protein biomarkers, or peptide biomarkers derived therefrom, present in the biological sample relative to the levels of the same proteins, or peptides derived therefrom, in a control sample; stratifying the risk of the patient at one or more time points for post-TBI anxiety when the measured levels of the one or more protein biomarkers, or peptide biomarkers derived therefrom, are increased or decreased in the subject's sample relative to the control levels; and if the risk stratification of the patient is high, administering psychotherapy or psychiatry or an effective amount of an anxiolytic depressant, such as Xanax, Librium, Klonopin, or Ativan to the patient.

In another embodiment of the invention, a method of treating post-TBI post-traumatic stress disorder (PTSD) includes: detecting whether a plurality of protein biomarkers in a protein biomarker panel, or peptide biomarkers derived therefrom, are present in a biological sample obtained from a patient; measuring the levels of the one or more protein biomarkers, or peptide biomarkers derived therefrom, present in the biological sample relative to the levels of the same proteins, or peptides derived therefrom, in a control sample; stratifying the risk of the patient at one or more time points for post-TBI PTSD when the measured levels of the one or more protein biomarkers, or peptide biomarkers derived therefrom, are increased or decreased in the subject's sample relative to the control levels; and if the risk stratification of the patient is high, administering psychotherapy or psychiatry to the patient.

In another embodiment of the invention, a method of treating post-TBI sleep disorder includes: detecting whether a plurality of protein biomarkers in a protein biomarker panel, or peptide biomarkers derived therefrom, are present in a biological sample obtained from a patient; measuring the levels of the one or more protein biomarkers, or peptide biomarkers derived therefrom, present in the biological sample relative to the levels of the same proteins, or peptides derived therefrom, in a control sample; stratifying the risk of the patient at one or more time points for post-TBI sleep disorder when the measured levels of the one or more protein biomarkers, or peptide biomarkers derived therefrom, are increased or decreased in the subject's sample relative to the control levels; and if the risk stratification of the patient is high, administering therapy at a sleep clinic or an effective amount of sleep aid, such as melatonin or Advil PM to the patient.

In another embodiment of the invention, a method of treating post-TBI headache includes: detecting whether a plurality of protein biomarkers in a protein biomarker panel, or peptide biomarkers derived therefrom, are present in a biological sample obtained from a patient; measuring the levels of the one or more protein biomarkers, or peptide biomarkers derived therefrom, present in the biological sample relative to the levels of the same proteins, or peptides derived therefrom, in a control sample; stratifying the risk of the patient at one or more time points for post-TBI headache when the measured levels of the one or more protein biomarkers, or peptide biomarkers derived therefrom, are increased or decreased in the subject's sample relative to the control levels; and if the risk stratification of the patient is high, administering an effective amount of analgesic, such as ibuprofen, acetaminophen to the patient.

In another embodiment of the invention, a method of treating post-TBI chronic pain includes: detecting whether a plurality of protein biomarkers in a protein biomarker panel, or peptide biomarkers derived therefrom, are present in a biological sample obtained from a patient; measuring the levels of the one or more protein biomarkers, or peptide biomarkers derived therefrom, present in the biological sample relative to the levels of the same proteins, or peptides derived therefrom, in a control sample; stratifying the risk of the patient at one or more time points for post-TBI chronic pain when the measured levels of the one or more protein biomarkers, or peptide biomarkers derived therefrom, are increased or decreased in the subject's sample relative to the control levels; and if the risk stratification of the patient is high, administering therapy from a pain specialist or an effective amount of analgesic, such as opioids or cannabidiols to the patient.

In another embodiment of the invention, a method of treating post-TBI oculomotor deficits includes: detecting whether a plurality of protein biomarkers in a protein biomarker panel, or peptide biomarkers derived therefrom, are present in a biological sample obtained from a patient; measuring the levels of the one or more protein biomarkers, or peptide biomarkers derived therefrom, present in the biological sample relative to the levels of the same proteins, or peptides derived therefrom, in a control sample; stratifying the risk of the patient at one or more time points for post-TBI oculomotor deficits when the measured levels of the one or more protein biomarkers, or peptide biomarkers derived therefrom, are increased or decreased in the subject's sample relative to the control levels; and if the risk stratification of the patient is high, administering vision therapy to the patient.

In another embodiment of the invention, a method of treating post-TBI attention and cognitive defects includes: detecting whether a plurality of protein biomarkers in a protein biomarker panel, or peptide biomarkers derived therefrom, are present in a biological sample obtained from a patient; measuring the levels of the one or more protein biomarkers, or peptide biomarkers derived therefrom, present in the biological sample relative to the levels of the same proteins, or peptides derived therefrom, in a control sample; stratifying the risk of the patient at one or more time points for post-TBI attention and cognitive defects when the measured levels of the one or more protein biomarkers, or peptide biomarkers derived therefrom, are increased or decreased in the subject's sample relative to the control levels; and if the risk stratification of the patient is high, administering cognitive therapy to the patient.

In another embodiment of the invention, a method of treating post-TBI balance and gait problems includes: detecting whether a plurality of protein biomarkers in a protein biomarker panel, or peptide biomarkers derived therefrom, are present in a biological sample obtained from a patient; measuring the levels of the one or more protein biomarkers, or peptide biomarkers derived therefrom, present in the biological sample relative to the levels of the same proteins, or peptides derived therefrom, in a control sample; stratifying the risk of the patient at one or more time points for post-TBI balance and gait problems when the measured levels of the one or more protein biomarkers, or peptide biomarkers derived therefrom, are increased or decreased in the subject's sample relative to the control levels; and if the risk stratification of the patient is high, administering physical therapy to the patient.

### Generation of Classification Algorithms for Qualifying Brain Injury Status

In some embodiments of the invention, data that are generated using samples such as “known samples” can then be used to “train” a classification model. A “known sample” is a sample that has been pre-classified. The data that are used to form the classification model can be referred to as a “training data set.” The training data set that is used to form the classification model may include raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., brain injury versus no brain injury).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Pat. Appl. Pub. No. 2002/0138208, which is incorporated in herein in its entirety.

In other embodiments of the invention, classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT Pub. No. WO 01/31580, U.S. Pat. Appl. Pub. No. 2002/0193950, U.S. Pat. Appl. Pub. No. 2003/0004402, and U.S. Pat. Appl. Pub. No. 2003/0055615, which are incorporated herein in their entireties.

Classification models can be formed on and used on any suitable digital computer. Suitable digital computers include micro, mini, or large computers using any standard or specialized operating system, such as a UNIX, WINDOWS® or LINUX™ based operating system. In embodiments utilizing a mass spectrometer, the digital computer that is used may be physically separate from the mass spectrometer that is used to create the spectra of interest, or it may be coupled to the mass spectrometer.

A training data set and classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including R, C, C++, visual basic, etc.

Learning algorithms, as described above, are useful both for developing classification algorithms for the biomarkers already discovered, and for finding new biomarker biomarkers. The classification algorithms, in turn, form the base for diagnostic tests by providing diagnostic values (e.g., cut-off points) for biomarkers used singly or in combination.

### Kits for the Detection of Biomarkers

In another aspect, embodiments of the invention provide kits for detecting, identifying, assessing, diagnosing, evaluating, or qualifying neurological injury or brain injury or the status thereof, e.g., qualifying TBI, mTBI or concussion, in a patient (subject). The kits are used to detect the protein biomarkers in the protein, or to detect a peptide derived therefrom. In a specific embodiment, the kit is provided as an ELISA kit including antibodies, or an antigen binding fragment thereof, that bind to one or more of, or a subset of, the plurality of protein biomarkers in protein biomarker panel, or a bindable peptide thereof.

The ELISA kit may include a solid support, such as a chip, microtiter plate (e.g., a 96-well plate), beads, or resin having protein biomarker capture reagents (e.g., binding molecules) attached thereon. The kit may further include a means for detecting the protein biomarkers, such as antibodies, and a secondary antibody-signal complex such as horseradish peroxidase (HRP)-conjugated goat anti-rabbit IgG antibody and tetramethyl benzidine (TMB) as a substrate for HRP.

The kit may be provided as an immuno-chromatography strip including a membrane on which specific antibodies are immobilized, and a means for detecting, e.g., gold particle bound antibodies, where the membrane, includes NC membrane and PVDF membrane. In certain embodiments, neurological injury or brain injury in a patient can be detected or diagnosed by adding to the kit a biological sample (e.g., blood or serum) obtained from the patient and detecting the relevant protein biomarkers that are bound to detectable antibodies, for example, by a method which includes: (i) collecting blood or serum from the patient; (ii) adding the blood or serum from the patient to the diagnostic kit; and, (iii) detecting the biomarkers bound to the antibodies. Use of the kit brings bound antibodies into contact with the patient's sample, such as blood or serum. If protein biomarkers of the protein biomarker panel (or peptides thereof) are present in the sample, the antibodies or antigen binding fragments thereof will bind to the proteins (or peptides thereof) in the sample and are detected. In other kit and diagnostic embodiments, blood or serum is not collected from the patient (i.e., it is already collected). In other embodiments, the sample may include a tissue sample or a clinical sample, which may be processed prior to contact with detection antibodies.

The kit can also include a washing solution or instructions for making a washing solution, in which the combination of the capture reagents and the washing solution allows capture of the biomarkers on the solid support for subsequent detection by, e.g., antibodies, such as by using an immunoassay or mass spectrometry. In a further embodiment, a kit can include instructions in the form of a label or separate insert. For example, the instructions may inform the user about how to collect the sample, and how to wash a support or substrate on which the particular biomarkers are bound and can be detected, etc. In yet another embodiment, the kit can include one or more containers with control biomarker samples, to be used as standard(s) or references for calibration or normalization.

The practice of the principles of the invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for certain embodiments will be discussed in the sections that follow.

Without further elaboration, it is believed that one skilled in the art, using the preceding description, can utilize the invention to the fullest extent. The following examples are illustrative only, and not limiting of the remainder of the disclosure or claims in any way whatsoever. In addition, the examples are put forth so as to provide those of ordinary skill in the art with a complete disclosure, description and exemplification of how to make and use the assay, screening, assessing, monitoring and therapeutic methods of the invention, and are not intended to limit the scope of what the inventors regard as their invention.

### EXAMPLES

Data disclosed herein relates to the utility of TBI biomarkers as indicators of persistent injury or pathology months after brain injury.

Mesoscale ECLIA discovery assays or colorimetric ELISAs were run to measure levels of the biomarkers in blood samples from healthy control subjects, orthopedic injury (or non-brain injury) control subjects, and mild TBI subjects. Measurements of blood biomarker protein levels at 6 months after injury were compared the biomarker protein levels obtained at the acute evaluation after injury, or with the biomarker protein levels of non-TBI control groups.

A subset of the HeadSMART study was used to evaluate biomarker levels in Examples 1 and 2. HeadSMART is a prospective study conducted at Johns Hopkins University, with serum sampling performed at initial evaluation, and at 7 subsequent time points up to 6 months post-injury. See Peters et al., Brain Injury, 2017, 31(3):370-378, which is incorporated by reference herein in its entirety by reference. Additional healthy controls enrolled at Baylor College of Medicine Ben Taub Hospital were also examined.

### Example 1

### Biomarker Elevations Determined During Acute Evaluation

Blood levels of the TBI biomarker proteins Glial Fibrillary Acidic Protein (GFAP), Brain Derived Neurotrophic Factor (BDNF), Neuron Specific Enolase (NSE, also called Enolase 2), Neurogranin (NRGN), von Willebrand Factor (vWF), soluble Suppressor of Tumorigenesis 2 (sST2 or ST2) were found to be significantly different in TBI subjects as compared to levels of the biomarkers in healthy control subjects and orthopedic injury control subjects. When the biomarker levels were determined at the first diagnostic evaluation of the orthopedic injury control subjects within 24 hours of orthopedic injury, the differences in the biomarker levels in comparison to levels of the biomarkers following confirmed TBI were less.

Longitudinal differences in paired blood samples from mild TBI patients (acute draw versus 6 months). Significant differences in blood levels of GFAP, BDNF, NSE, NRGN, vWF, and ST2 were observed in paired TBI subjects when levels of the biomarkers were measured at the initial TBI evaluation within 24 hours of brain injury to the levels of the biomarkers in the same subjects 6 months after injury (chronic TBI). When restricting attention to independent subjects (acute versus chronic), significant differences between the blood levels of GFAP, BDNF, NRGN, VWF, ST2, and NSE were observed at the initial time of evaluation of TBI subjects within 24 hours of injury in comparison to the levels of these biomarkers in the same subjects 6 months after injury.

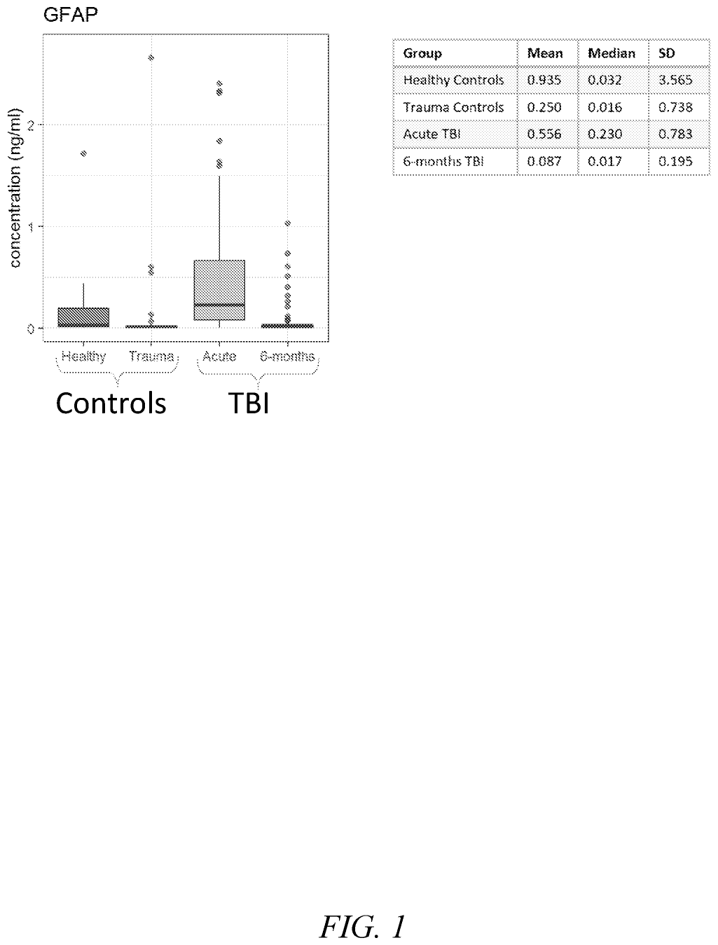

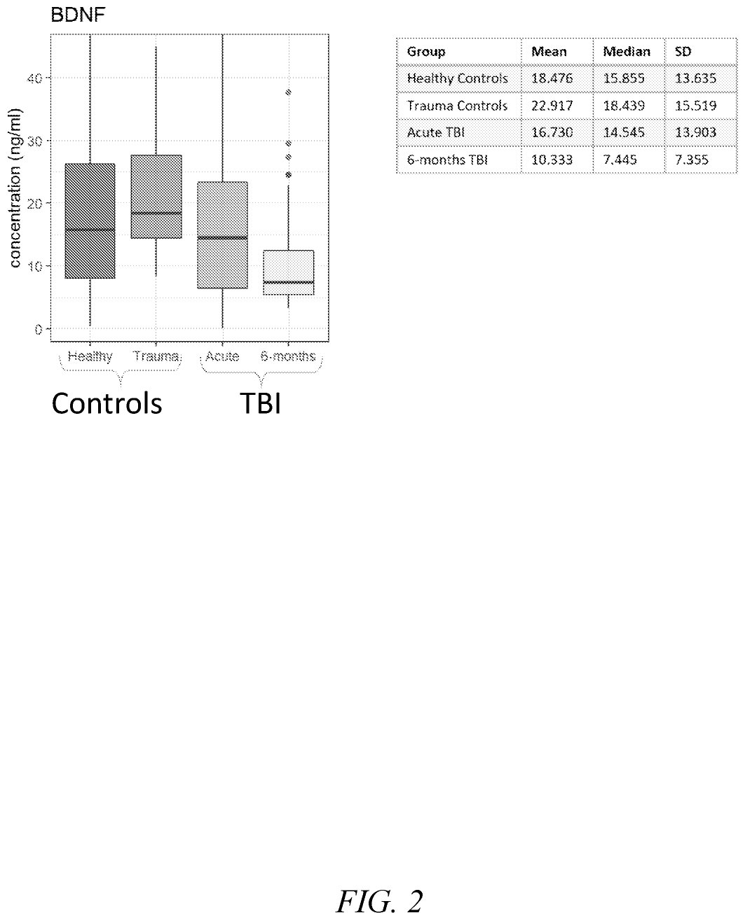

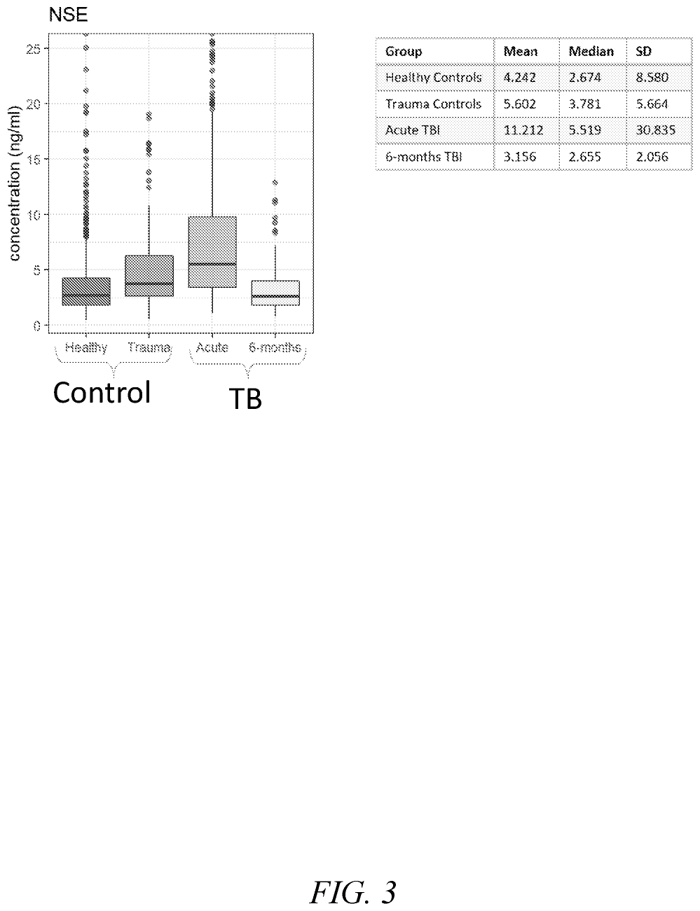

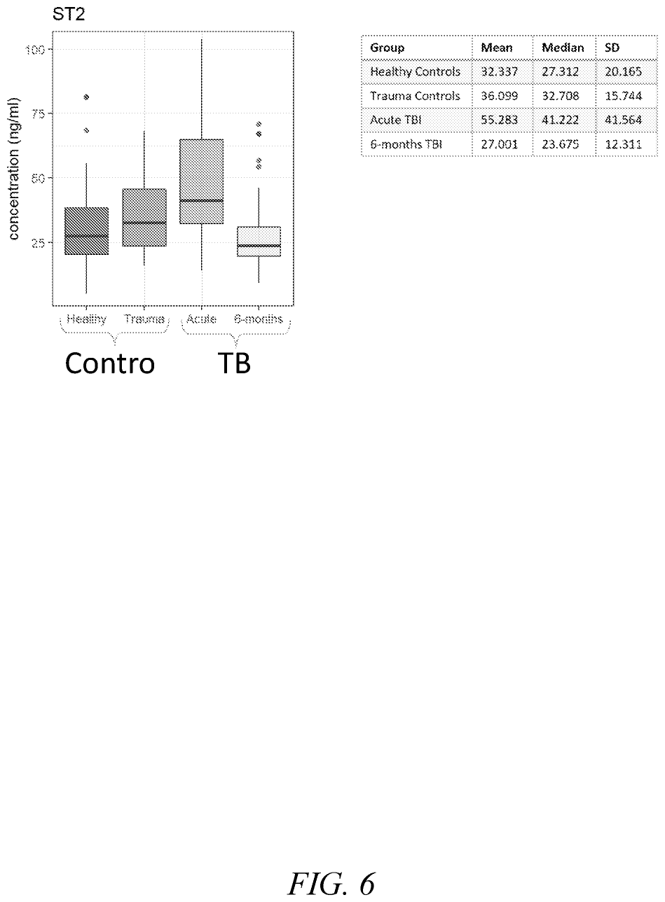

GFAP, BDNF, NSE, and ST2 were significantly higher at the first acute evaluation, compared with levels detected at 6 months. See FIGS. 1, 2, 3, and 6, respectively.

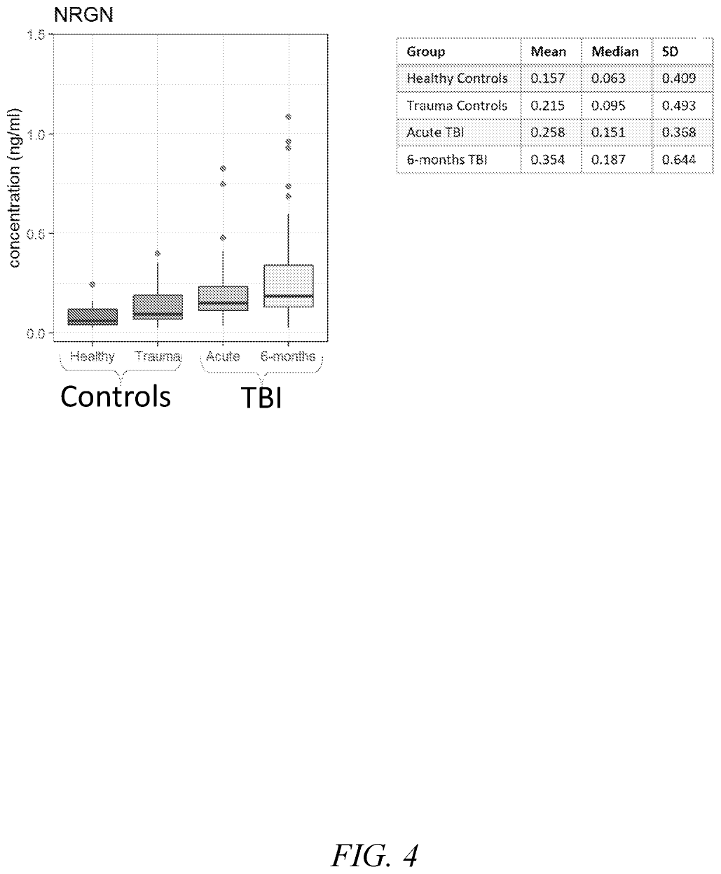

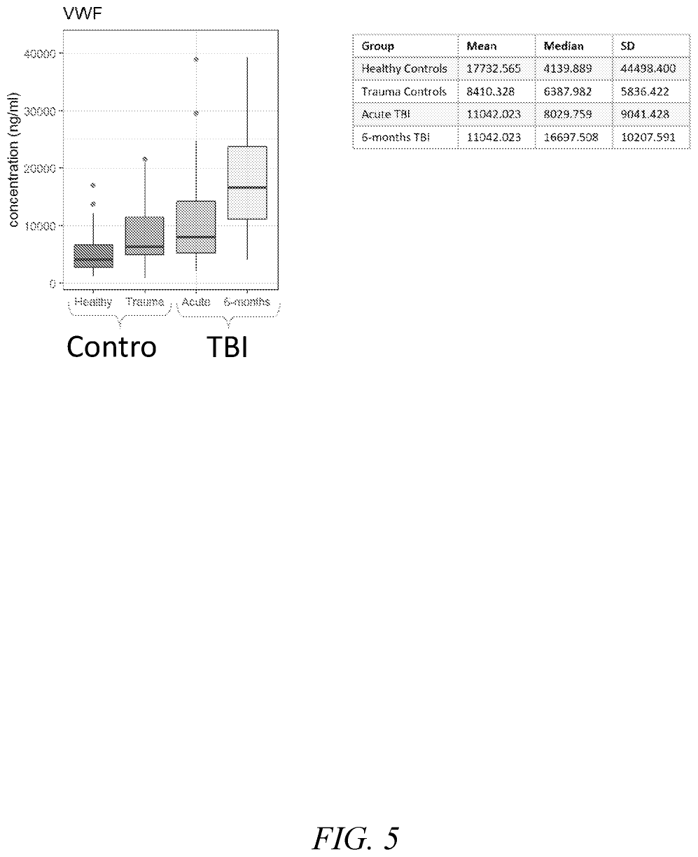

NRGN and vWF were elevated after acute injury, but increased further between acute evaluation and 6 months. These increases were significant. See FIGS. 4 and 5, respectively.

Chronic changes in BDNF for patient monitoring purposes. BDNF levels differed in TBI patients when measured in the blood at 6 months from injury in patients that had acute amnesia compared to those without acute amnesia. (P=0.033), and also in patients who had focal neurological deficits at the acute evaluation.

Soluble ST2 was also found to be significantly different at 6 months following injury in males versus females (P=0.009).

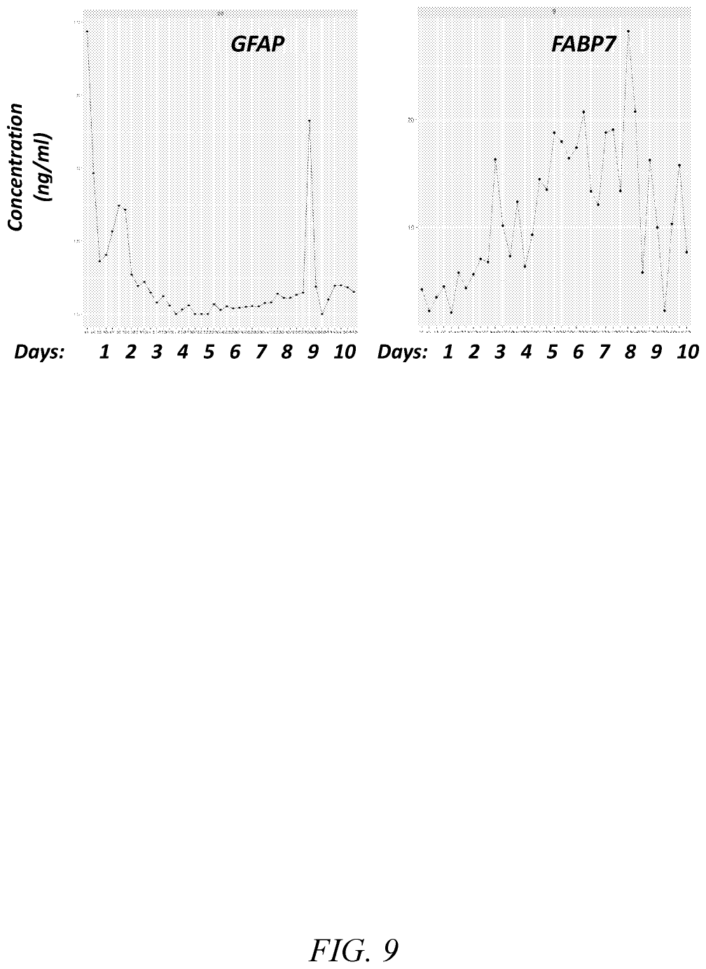

Fatty acid binding protein 7 (FABP7) levels in severe TBI. After an initial elevation in blood levels of FABP7 in acute, severe TBI subjects within 3 days of brain injury, FABP7 levels were observed to decrease and then increase again during the following 2 to 3 days. See FIG. 9. By contrast, in the same subjects, GFAP levels were significantly elevated within 24 hours of brain injury and then dropped nearly to control levels within 4 days.

### Example 2

Biomarker Levels and Long-Term Outcomes after TBI and Diagnostic Uses for Long-Term Monitoring of Ongoing Pathobiology

The association between blood biomarker levels and brain injury-related outcomes was studied to identify statistically significant associations (comparing levels of biomarkers in TBI Symptomatic vs. Asymptomatic groups). The following associations meet the α=0.05 level of significance with no adjustment for multiple comparisons.

Global outcome measures-Disability and combined symptoms at 1, 3, or 6 months. Glasgow Outcome Scale Extended (GOS-E) and the Post-concussive symptom score (PCS, based on the Rivermead post-concussion questionnaire RPQ16 or the ICD-10 codes) are measures of overall disability and symptom burden, respectively. These are commonly used clinical outcome measures.

GFAP blood levels detected at the acute injury evaluation within 24 hours of brain injury were significantly different in TBI subjects that experienced incomplete recoveries due to a lasting disability at 1 month (p=0.01) or 3 month (p=0.02) assessments. These results indicated that GFAP could be used as a predictor of recovery outcome. The group assessments were parsed by GOS-E score (GOS-E score <8 versus 8 at each assessment time point) by gose1m (p=0.01), gose3m (p=0.02), icd-10pcs1m (p=0.017), pcs1m (p=0.017).

Symptom category—Affective Anxiety Disorders-Neuropsychiatric. BDNF measured at acute evaluation (up to 24 h from injury), differed by Generalized Anxiety Disorder 7 (GAD7) status (Symptomatic=score of 10 or greater, indicating moderate to severe anxiety symptoms) at 6 months after injury (p=0.018).

NSE measured at acute evaluation (up to 24 h from injury), differs by GAD7 status (Symptomatic=score of 10 or greater, indicating moderate to severe anxiety symptoms) at 3 months after injury (3m outcome) (p=0.025).

ST2 levels measured at acute evaluation (up to 24 h from injury), differed by GAD7 status (Symptomatic=score of 10 or greater, indicating moderate to severe anxiety symptoms) at 1 month after injury (P=0.037).

Symptom category: Depressive symptoms Disorders—vWF Chronic vWF elevation, as measured at 6 months after injury, was found to differ by PHQ9-defined depressive symptom status at 3 months after injury. In patients with a score of 10 or greater, which indicates moderate to severe depressive symptoms (p=0.003), vWF remained elevated months after injury.

### Example 3

### Differential Detection of Biomarkers at Acute and Chronic TBI Phases

Data from 34 patient blood samples taken from 17 Traumatic brain injury (TBI) subjects, who met the criteria for Mild TBI were studied to assess relationships between blood levels of protein biomarkers of blood brain barrier (BBB) vascular leakage and structural alterations to the BBB, at acute and chronic stages of TBI, as described below.

Blood levels of the following biomarkers were assessed: von Willebrand Factor (vWF); the vWF A2 fragment (vWF2); Brain-Derived Neurotrophic Factor (BDNF); Glial Fibrillary Acidic Protein (GFAP); and Neurogranin (NRGN). Biomarker levels were assessed in blood samples drawn acutely (within 3 days) and at 6 months after brain injury using Mesoscale Discovery (MSD) electrochemiluminescence immunoassays. These assays utilize a sandwich immunoassay format, which gains signal specificity from two antibodies, one being a capture antibody and the other, a detection antibody, to derive signal, which is quantified compared to a calibrant dilution curve that relates signal to quantity of analyte present in the sample. Detected levels of each biomarker at the acute and chronic stages of TBI were used to calculate ratios of chronic levels to acute levels. These ratios are called C-ratios. A C-ratio of greater than 1 indicates an elevated level of a biomarker in a subject with chronic TBI.

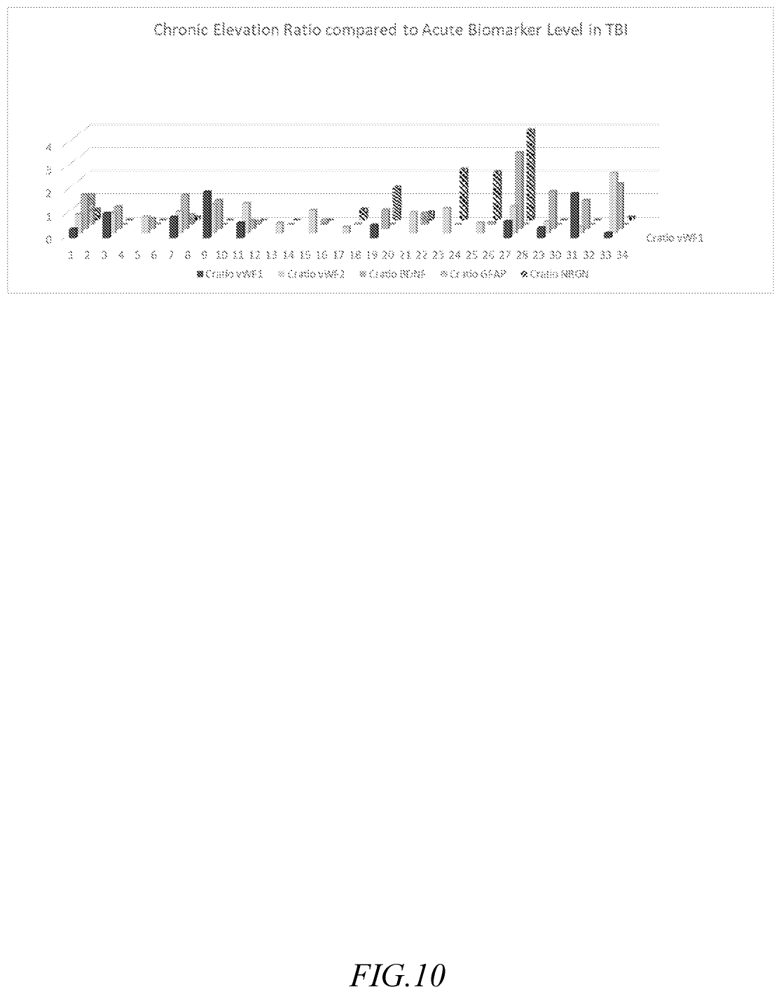

These data explored the chronic elevation status across the biomarkers studied in the subjects. FIG. 10 plots C-ratios for vWF1, vWF2, BDNF, GFAP and NRGN. It was observed that, while GFAP is not elevated (all C-ratios <1), NRGN in particular is highly elevated at 6 months post-TBI in some patients, and to some extent vWF1, vWF2, and BDNF were also elevated at 6 months post-TBI. It can be appreciated that not all biomarkers were elevated in the same samples, which reflected the heterogeneity that is typically observed among cases of mild TBI, because it is derived from different locations of damage to the brain and mechanisms of injury, as well as individual response to injury. Median levels of the biomarker proteins vWF1, vWF2, BDNF, and NRGN, but not GFAP were all found to be elevated at a chronic stage as compared to their levels at the acute stage of TBI.

As stated above, and shown in FIG. 10, GFAP is not prominent among elevated biomarkers in comparison with vWF1, vWF2, BDNF and NRGN. By contrast, NRGN levels were elevated at the acute stage, but were then expected to normalize back to healthy ranges of levels after repair and recovery of the central nervous system (CNS).

Based on the foregoing results, C-ratios of the tested biomarkers can be used to identify individuals with ongoing TBI pathology. Similarly, ratios of detected levels of the same biomarkers in healthy subjects (i.e., subjects without a TBI) to levels of the biomarkers in subjects that potentially have a chronic TBI may also be used to identify individuals with ongoing TBI pathology. Such information would support a method for identifying the need for follow-up TBI assessments. To that end: Table 1A provides ratios of the levels of the listed biomarkers in blood drawn from subjects with chronic TBI (6 months post-TBI) to mean healthy control levels of the biomarkers; and Table 1B provides C-ratios based on the same data in FIG. 10. A significant elevation of a biomarker level was considered to be a >1 ratio for comparisons to healthy control levels, and a >0.5 ratio for comparisons of chronic to acute biomarker levels. Elevated ratios in chronic TBI subjects are in bold underlined text.

TABLE 1A
Biomarker Level Ratios: Chronic to Mean Healthy Control

| | Chronic | Chronic | Chronic | Chronic |
|---|---|---|---|---|
| | to healthy | to healthy | to healthy | to healthy |
| Subject | vWF | BDNF | GFAP | NRGN |
| Number | ratio | ratio | ratio | ratio |
| 1 | 0.695746 | 1.748671 | 6.956896 | 0.770073 |
| 2 | 0.649533 | 0.638437 | 0 | 1.311074 |
| 3 | 0.432212 | 0.788279 | 0.060108 | 0 |
| 4 | 0.525645 | 1.385102 | 0.680925 | 1.839838 |
| 5 | 0.389017 | 1.367653 | 0 | 0 |
| 6 | 0.287894 | 0.471913 | 0.025537 | 0 |
| 7 | 0.252219 | 0.40058 | 0.257147 | 1.171747 |
| 8 | 0.720612 | 1.069538 | 0.222494 | 1.554989 |
| 9 | 0.770462 | 1.555334 | 0.032121 | 0 |
| 10 | 0.494431 | 2.033823 | 0 | 0 |
| 11 | 0.270031 | 2.587051 | 0 | 0.446011 |

TABLE 1B
Biomarker: Ratio of Chronic to Acute

| | Chronic | Chronic | Chronic | Chronic |
|---|---|---|---|---|
| Subject | to Acute | to Acute | to Acute | to Acute |
| Number | vWF | BDNF | GFAP | NRGN |
| 1 | 0.832657 | 1.47829945 | 1.28706652 | 0.477542 |
| 2 | 0.877054 | 0.95929255 | 0 | 0 |
| 3 | 0.722268 | 0.44510669 | 0.0206179 | 0 |
| 4 | 0.944409 | 1.47229085 | 0.42182897 | 0.13971 |
| 5 | 1.247992 | 1.23914182 | 0 | 0 |
| 6 | 1.307263 | 0.36708748 | 0.14763926 | 0 |
| 7 | 0.44741 | | 0 | 0.009046 |
| 8 | 1.001093 | | 0.19677338 | 0 |
| 9 | 0.275939 | | 0.03030175 | 0.480813 |
| 10 | | 0.83149936 | 0.02068072 | 1.433435 |
| 11 | 0.916342 | | 0.49403418 | 0.345967 |
| 12 | 1.085123 | | 0 | 2.198538 |
| 13 | 0.450481 | | 0.08989063 | 2.086567 |
| 14 | 1.162556 | 3.2945306 | 0.02224395 | 3.891589 |
| 15 | 0.511879 | 1.62465852 | 0.00276162 | 0 |
| 16 | 0.284731 | 1.23644726 | 0 | 0 |
| 17 | 2.602675 | 1.94201979 | 0 | 0.128561 |

Correlations of Chronic TBI Biomarkers with Outcomes and Structural Pathology

Structural pathology can remain after TBI and may be the cause or effect of ongoing abnormal processes. For example, glial scarring can be a cause of ongoing functional changes or seizures resulting from different types of injury, including injuries resulting from surgery, viral injections, trauma, and tumors. The delicate microvasculature in the brain is dense and susceptible to injury, such that even milder TBI subjects may have widespread microvascular injury that is beyond the sensitivity of computerized tomography (CT) scans to detect. The high metabolic activity of the brain relies on this microvasculature to function. Imperfect healing of the BBB along this microvasculature that results in a breach of the BBB is common after a brain injury. The breach allows blood proteins and other contaminants to leak into the brain, which, in turn, causes local inflammation. A technique, like dynamic contrast enhanced MRI (DCE-MRI), can be used to provide an important readout on the functional status of the vasculature throughout the brain following TBI.

Structural findings made using DCE-MRI showed relationships between chronic alterations in TBI biomarker levels and BBB leakage, by quantifying the amount of contrast agent that escapes the blood stream into the brain after intravenous administration of the dye. This technique, which demonstrates an anatomical defect remaining from improper healing of the brain vasculature after TBI, can be quantified by published metrics, including the NPI index and K^{trans}. Both indices measure changes in blood flow over time and are highly correlated to chronic symptoms of the TBI pathology, including neuropsychiatric changes, seizures, and neurodegenerative processes. These methods were used to correlate differences in blood levels of the TBI biomarkers at acute and chronic stages of TBI with brain neurovascular pathology. Those correlations are reported in Table 2, which shows calculations of the correlation coefficients based on comparisons of C-ratios of vWF, BDNF, GFAP, and NRGN to Normalized permeability Index (NPI) values, which, here, are measures of ongoing blood brain barrier damage detected by DCE_MRI neuroimaging. These data showed that chronic NRGN elevation was positively correlated with vascular pathology at 6 months after TBI. This correlation supports a role for NRGN testing as an indicator of ongoing neurovascular pathology that could involve advanced imaging or other tests to better understand the patient's pathology and make treatment decisions for related chronic symptoms.

TABLE 2

| | Correlation |
|---|---|
| Correlation | coefficient |
| NPIr-VWFcr* | −0.41782 |
| NPIr-BDNFcr* | 0.087887 |
| NPIr-GFAPcr* | 0.027257 |
| NPIr-NGRNcr* | 0.67398 |

*Definitions for comparisons: NPIr (Normalized Permeability Index ratio), 6 month-NPI/Acute NPI; VWFcr, chronic ratio for level/acute vWF level; BDNFcr, BDNF chronic/acute level; GFAPcr, GFAP chronic/acute ratio; NRGNcr, NRGN chronic/acute ratio. Significant correlation coefficient is indicated by bold and underlined text.

What is claimed:

1. A method of determining ongoing neurodegenerative and/or ongoing neurovascular dysfunction in a subject known to have had or suspected of having had a traumatic brain injury (TBI) event, the method comprising:
(A) obtaining a biological sample from the subject;
(B) measuring the level of the biomarker, von Willebrand Factor (vWF), and one or more of the additional biomarkers in the biological sample:
(i) Neurogranin (NRGN);
(ii) Brain Derived Neurotrophic Factor (BDNF); and
(iii) Fatty acid binding protein 7 (FABP7); and;
(C) determining ongoing neurodegenerative dysfunction and/or ongoing neurovascular dysfunction in the subject when the measured levels of vWF and one or more of the additional biomarkers are altered relative to respective reference levels of the biomarkers.

2. The method of claim 1, wherein ongoing neurodegenerative dysfunction is determined when the measured level of NRGN is increased and at least one of BDNF and FABP7 is decreased relative to the respective reference levels of same one or more biomarkers.

3. The method of claim 1 wherein ongoing neurovascular dysfunction is determined to be indicative of chronic TBI based upon the time period after injury in which the sample was obtained.

4. The method of any of claims 1-3, wherein the respective reference levels of the biomarkers were determined using a biological sample obtained from the same subject at least about three (3) months prior to obtaining the biological sample in step (A), thereby indicating chronic TBI.

5. The method of claim 1, wherein the reference levels were determined within 24 hours of the traumatic brain injury event.

6. The method of any one of claims 1-5, wherein ongoing neurodegenerative and/or ongoing neurovascular dysfunction in the subject is determined when a ratio of the level of vWF and one or more of the additional biomarkers in the biological sample to the reference levels of the vWF and the same one or more additional biomarker is more than about 1.

7. The method of claim 6, wherein the ratio is more than about 2 or more than about 3.

8. The method of any of claims 1-7, further comprising administering treatment for a brain injury when altered levels of vWF and one or both of NRGN and BDNF are detected.

9. A method of monitoring a neurodegenerative and/or neurovascular dysfunction in a subject known to have had, or suspected of having had a traumatic brain injury (TBI), the method comprising:
(A) obtaining first and second biological samples from the subject at first and second timepoints, respectively, wherein the second timepoint is after the first timepoint;
(B) measuring the level of the biomarker, von Willebrand Factor (vWF), and one or more of the additional biomarkers in the biological sample:
(i) Neurogranin (NRGN);
(ii) Brain Derived Neurotrophic Factor (BDNF); and
(iii) Fatty acid binding protein 7 (FABP7); and
(C) administering treatment for TBI to the subject when the measured level of VWF is increased and the level of one or more of the additional biomarkers are altered in the second sample relative to the first sample.

10. The method of claim 9, wherein the level of NRGN is increased.

11. The method of claim 9 or 10, wherein the level of one or more of the additional biomarkers is decreased.

12. The method of claim 9, wherein ongoing neurodegenerative dysfunction is determined when the measured levels of NRGN are increased and BDNF remains decreased in the second sample relative to the levels of the same biomarkers in the first sample.

13. The method of claim 9, wherein ongoing neurovascular dysfunction, as a manifestation of chronic TBI, is indicated by increased biomarker levels in vWF and one or more of Neurogranin, NSE, GFAP, or ST2 or decreased in one or more of BDNF or FABP7, based upon the time period after injury in which the second sample was obtained relative to the time the first sample was obtained.

14. The method of any one of claims 9-13, wherein the first timepoint is about 3 months, about 4 months, about 5 months, about 6 months, about 7 months, about 8 months, about 9 months, about 10 months, about 11 months, or about 12 months prior to obtaining the biological sample in step (A), thereby indicating chronic TBI.

15. The method of claim 14, wherein the first timepoint is within 24 hours of the traumatic brain injury that the subject known to have had or suspected of having had.

16. The method of any one of claims 9-15, wherein treatment for TBI is administered to the subject when a ratio of the levels of vWF and one or more of the additional biomarkers in the second sample to the levels of vWF and the same one or more additional biomarkers in the first sample is more than about 1.

17. The method of claim 16, wherein the ratio is more than about 2 or more than about 3.

18. The method of claim 16 or 17, further comprising imaging the brain of the subject by dynamic contrast enhanced MRI (DCE-MRI) to generate normalized permeability index (NPI) values on or about the same days as the first and second timepoints, and administering treatment for TBI when the biomarker value generated on or about the second timepoint is elevated relative to the biomarker value generated on or about the first timepoint.

19. A method of monitoring a secondary brain injury in a subject known to have had, or suspected of having had a traumatic brain injury (TBI), the method comprising:
(A) obtaining first and second biological samples from the subject at first and second timepoints, respectively, wherein the second timepoint is after the first timepoint;
(B) measuring the level of the biomarker, von Willebrand Factor (vWF), and one or more of the additional biomarkers in the biological sample:
(i) Neurogranin (NRGN);
(ii) Brain Derived Neurotrophic Factor (BDNF); and
(iii) Fatty acid binding protein 7 (FABP7); and
(C) administering treatment for TBI to the subject when the measured levels of vWF and the level of one or more of the additional biomarkers are altered in the second sample relative to the first sample.

20. The method of claim 19, wherein the level of NRGN is increased.

21. The method of claim 19 or 20, wherein the level of one or more of the additional biomarkers is decreased.

22. The method of claim 19, wherein ongoing neurodegenerative dysfunction is determined when measured levels of NRGN are increased, and BDNF are decreased in the second sample relative to the levels of the same biomarkers in the first sample.

23. The method of claim 19, wherein ongoing neurovascular dysfunction is determined to be indicative of chronic TBI based upon the time period after injury in which the second sample was obtained relative to the time the first sample was obtained.

24. The method of any one of claims 19-23, wherein the first timepoint is about 3 months, about 4 months, about 5 months, about 6 months, about 7 months, about 8 months, about 9 months, about 10 months, about 11 months, or about 12 months prior to obtaining the biological sample in step (A).

25. The method of claim 24, wherein the first timepoint is within 24 hours of the secondary traumatic brain injury that the subject known to have had or suspected of having had.

26. The method of any one of claims 19-25, wherein treatment for TBI is administered to the subject when a ratio of the levels of vWF and one or more of the additional biomarkers in the second sample to the levels of vWF and the same one or more additional biomarkers in the first sample is more than about 1.

27. The method of claim 26, wherein the ratio wherein the ratio is more than about 2 or more than about 3.

28. The method of any one of claims 1-8, wherein the measuring step, (B), further comprises measuring the levels of one or more of the biomarkers Glial Fibrillary Acidic Protein (GFAP), Neuron Specific Enolase (NSE), and Suppressor of Tumorigenesis 2 (ST2) in the biological sample and determining differences in the levels of the one or more biomarkers relative to respective reference levels of the biomarkers.

29. The method of any one of claims 9-28, wherein the measuring step, (B), further comprises measuring the level of one or more of the biomarkers Glial Fibrillary Acidic Protein (GFAP), Neuron Specific Enolase (NSE), and Suppressor of Tumorigenesis 2 (ST2) in the first and second biological samples and determining a difference in the level of the one or more biomarkers in the second sample relative to the level of the same one or more marker in the first sample.

30. The method of any one of claims 1-29, wherein the measuring step, (B), comprises:
(i) contacting the biological sample with a plurality of antibodies or antibody fragments specific for vWF and a plurality of antibodies or antibody fragments specific for one or more of the biomarkers NRGN, BDNF, and FABP7,
respectively; and
(ii) detecting binding of the vWF-specific antibodies or antibody fragments specifically bound to vWF and detecting binding of the one or more additional biomarker-specific antibodies or antibody fragments specifically bound to one or more additional biomarker in the sample to measure the levels of the biomarkers.

31. The method of claim 29, wherein the measuring step, (B), further comprises:
(i) contacting the biological sample with a plurality of antibodies or antibody fragments specific for one or more of the biomarkers GFAP, NSE, and ST2, respectively; and
(ii) detecting binding of antibodies or antibody fragments specifically bound to the biomarkers in the sample to measure the levels of the biomarkers.

32. The method of claim 30 or 31, wherein the step of detecting binding of the antibodies or antibody fragments is carried out by an immunoassay, an immunoblotting method, an immunoprecipitation assay, an immunostaining method, a quantitative assay, an immunofluorescent assay, or a chemiluminescence assay.

33. The method of any one of claims 1-29, wherein the measuring step, (B), comprises using mass spectroscopy to measure levels of one or more biomarkers.

34. The method of any of claims 1-33, wherein the biological samples are blood, plasma, serum, cerebrospinal fluid (CSF), saliva, or tissue.
